(19) United States
(12) Patent Application Publication
(10) Pub. No.: US 2016/0030857 A1
(43) Pub. Date: Feb. 4, 2016

(54) Component Recovery Process

(71) Applicant: INVISTA North America S.a r.l., Wilmington, DE (US)

(72) Inventors: Anne Gaffney, West Chester, PA (US); James D. Hastings, Victoria, TX (US); Frank E. Herkes, Wilmington, DE (US); Milind V. Kantak, Wilmington, DE (US); Robert B. Osborne, Wilmington, DE (US); Richard W. Pearlstein, Newark, DE (US)

(73) Assignee: INVISTA North America S.a r.l., Wilmington, DE (US)

(21) Appl. No.: 14/805,621

(22) Filed: Jul. 22, 2015

Related U.S. Application Data
(60) Provisional application No. 62/032,802, filed on Aug. 4, 2014.

(51) Int. Cl. B01D 3/06; C07C 51/48; B01D 3/40

(52) U.S. Cl. CPC B01D 3/06; B01D 3/40; C07C 51/48

(57) ABSTRACT

The present disclosure provides a method for separating a mixture comprising a compound of formula I:

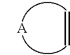

and a compound of formula III:

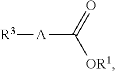

comprising distilling the mixture, wherein A is a C₆-C₁₀ alkene chain with at least one double bond, R¹ is a C₁-C₁₀ alkyl, and R³ is an oxygen-containing functional group.

### CROSS-REFERENCE TO RELATED APPLICATIONS

This application claims the priority filing date of U.S. Provisional application Ser. No. 62/032,802, filed on Aug. 4, 2014, the disclosures of which are specifically incorporated herein by reference in their entireties.

### BACKGROUND OF THE INVENTION

It is known that 1,5,9-cyclododecatriene (CDDT) and 1,5-cyclooctadiene (COD) are co-products of the cyclotrimerization of butadiene, and that each are available on an industrial scale. The conversion of CDDT and COD to multi-functionalized acyclic compounds has immediate industrial importance as a source of additives, intermediates and monomers.

International Application Publication No. WO2015/006360A1 (“the '360 Publication”), filed Jul. 8, 2014, discloses an ozonolysis method of making a compound of formula IIa:

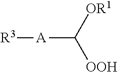

from a compound of formula I:

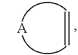

wherein A is a C₆-C₁₀ alkene chain with at least one double bond, R¹ is a C₁-C₁₀ alkyl, and R³ is an oxygen-containing functional group.

The compound of formula I may include cyclic trienes and cyclic dienes (e.g., CDDT and COD).

As disclosed in the '360 Publication, the ozonolysis reaction can be conducted under conditions to selectively ozonize only one carbon-carbon double bond in the compound of formula I to form the compound of formula IIa. In a non-selective ozonolysis, more than one carbon-carbon double bonds are converted and non-selective products are formed. In some embodiments, the ozonolysis conditions favor the compound of formula IIa with the preservation of “A” as in the compound of formula I. For example, in the compound of formula IIa from the ozonolysis of CDDT, “A” should be a C₁₀ alkene chain with two carbon-carbon double bonds. Due to the cleaving of more than one double bonds, in some embodiments, the non-selective products contain the compound of formula IIa with fewer carbon numbers in “A” than the compound of formula I.

In some embodiments, the ozonolysis effluent may comprise from about 0 wt. % to about 50 wt. % the compound of formula I, from about 0 wt. % to about 80 wt. % reagent, from about 0 wt. % to about 50 wt. % the compound of formula IIa, and up to about 15 wt. % non-selective products. The non-selective products may include compounds having two terminal oxygenated groups, which include dialdehydes, diacids, diesters, acid-esters, aldehyde-acids. In some embodiments, at least some of the non-selective products are saturated, for example, linear C₄ species. In a preferred embodiment, the ozonolysis effluent comprises from about 0 wt. % to 50 wt. % of the compound of formula I, from about 0 wt. % to about 80 wt. % reagent, from about 0 wt. % to about 50 wt. % the compound of formula IIa, and up to about 10 wt. % non-selective products. In some embodiments, the ozonolysis effluent is a stable, flowable liquid at ambient conditions.

International Application Publication No. WO2015/073672 A2 (“the '672 Publication”), filed Nov. 13, 2014, further discloses a process for transforming the compound of formula IIa:

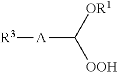

to a compound of formula III:

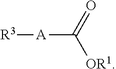

The disclosures of the '360 Publication and the '672 Publication are specifically incorporated herein by reference in their entireties.

The transformation may be in the presence of an acid anhydride. Examples of suitable anhydrides include, but are not limited to, acetic anhydride (“ACAN”), succinic anhydride, maleic anhydride, other anhydrides belonging to the general anhydride family and mixtures thereof. Acetic anhydride is preferred.

Also, the transformation may be in the presence of a catalyst, e.g., a mixture of acid and amine, which can be freshly mixed, premixed, azeotropically co-distilled, or recycled.

Examples of suitable acids include, but are not limited to, acetic acid, succinic acid, maleic acid. Acetic acid is preferred. Examples of suitable amines include, but are not limited to, triethyl amine, diethanol amine, tributyl amine, pyridine, other unsubstituted or substituted amines belonging to the general amines family and mixtures thereof. Triethyl amine is preferred.

The crude reaction mixture from the ozonolysis transformation reaction of the compound of formula I may comprise the compound of formula III, the compound of formula I, solvent (optional), ACAN, acetic acid, and triethylamine.

From an industrial standpoint, it would desirable to recover the unutilized components for economic value in conjunction with concentrating the target product(s) for downstream processing. Through intensive research, inventors realize that the complex vapor-liquid and liquid-liquid interactions of the components present in this effluent make the component separation very demanding while preserving the main ozonolysis transformation product, e.g., the compound of formula III, from thermal degradation.

In fact, the components present in the effluent may have tendencies to form azeotropes with each other in various combinations that make the separation even more complex to handle. Common industrial practices of dealing with such indigenous complex, azeotrope-forming mixtures are: to spend the capital for multi-distillation equipment, using special unit operations such as solvent extraction, absorption, membrane separation, and/or combinations thereof. Such complicated separations become overall costly. The recovered component yields also generally suffer from such complex techniques; not to mention the cost and complexity of having to manage the extraction solvents that are employed to disrupt the azeotrope stability during the extraction process.

It would be of considerable economic importance if a cost-effective process could be developed for the recovery of unutilized components present in the above-mentioned reaction mixture. A practical method for the separation of the multi-component mixture, obtained from the transformation process, is a basic requirement, both, for economic and engineering reasons.

U.S. Pat. No. 3,059,028 to Robert H. Perry (the '028 patent) discloses a process for the conversion of a cyclic triolefin via selective monoozonolysis to provide an olefinic monoozonolysis product. The process employs a cyclic non-conjugated polyolefin, a reactive ozonolysis solvent, an unreactive ozonolysis solvent, or a mixture thereof. At the end of the ozonolysis reaction, the reaction mixture is said to contain solvent, monoozonolysis product, and unreacted polyolefin. In Example I of the '028 patent, a method of recovering the monoozonolysis product from the reaction mixture includes room-temperature evaporation of the solvent mixture under a reduced pressure to provide two liquid phases. Further extraction with another solvent recovers the peroxidic monoozonolysis product assisted by excess methanol. The isolated peroxidic monoozonolysis product is obtained upon methanol evaporation under a reduced pressure.

As described above, one disadvantage of the '028 patent Example I is the rather complicated peroxidic monoozonolysis product recovery from the reaction mixture, which requires multiple extraction steps of solvent additions and stripping of the same under reduced pressure.

The disclosed process eliminates the multiple solvent extractions and stripping steps; hence making the improved separation process more cost advantaged. Furthermore, the disclosed process affords a reasonably complete separation between all components that may be recovered, reclaimed or recycled back into the process. The effectiveness and practical simplicity of the disclosed process towards managing the aforementioned multi-component mixture may be appreciated by those skilled in the field.

Schreiber et. al., in Tetrahedron Letters, Vol. 23, No. 38, 3867 (1982), describe a method to ozonolytically cleave an olefin. The ozonolysis of Schreiber affords an aldehyde-alkoxy hydroperoxide.

Schreiber does not provide the purification scheme for the obtained one-pot sequence mixture. To be of commercial value, a need exists for a practical method to efficiently: (i) concentrate the target product, (ii) recover the unutilized components, and (iii) purge out the undesired impurities. The Schreiber one-pot sequence preparations fail to meet this need from an industrial standpoint.

Dygos et al., in J. Org. Chem. 1991, 56, 2549-2552, disclose an 11-step synthesis of the antisecretory prostaglandin enisoprost starting with (Z,Z)-1,5-cyclooctadiene. Methyl 8-oxo-4(Z)-octenoate was synthesized and purified by a rather complicated system, which involves multiple reagents, complicated reaction conditions, and multiple filtrations and extractions to obtain a crude product with about 80% purity.

In addition, Dygos et al. do not disclose a method for the recovery of other process materials from the reaction mixture. The disclosed process addresses this need for efficiently and cost-effectively recovering the unutilized components from the reaction mixture, making the disclosed process commercially advantageous.

U.S. Pat. No. 4,085,127 to SNIA Viscosa (the '127 patent) gives a method for producing aldehyde acids by selective ozonization of cyclo-olefins. An installation, schematically shown in FIG. 1 of the '127 patent, is made up of twenty-one unit operations and involves multiple unit operations for component recovery.

In view of the disadvantages stated above, there exists a need for the development of scalable separation methods from a profitable industrial application standpoint.

Accordingly, it will be desirable to provide a process which performs, in a technically simple and economically viable manner, the unit operations, separation stages and treatments under conditions which may be well suited in the commercial field, more particularly, in an industrial scale processing with easily attainable and controllable process conditions.

### SUMMARY OF THE INVENTION

One aspect of the disclosed process is directed to a method for separating a mixture comprising a compound of formula I:

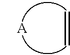

and a compound of formula III:

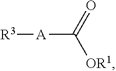

comprising distilling the mixture, wherein A is a C₆-C₁₀ alkene chain with at least one double bond, R¹ is a C₁-C₁₀ alkyl, and R³ is an oxygen-containing functional group.

Another aspect of the disclosed process is directed to a method of separating a miscible mixture comprising a compound of formula I:

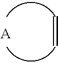

and an acid into two phases, comprising adding a phase-separation agent to the mixture, wherein A is a C₆-C₁₀ alkene chain with at least one double bond.

### BRIEF DESCRIPTION OF THE FIGURES

FIG. 1 shows a diagrammatic flow of the disclosed multi-component mixture separation scheme according to one embodiment.

FIG. 2 is a representation of one embodiment of separation section 141 shown in FIG. 1.

FIG. 3 is a representation of one embodiment of separation section 105 shown in FIG. 1.

FIG. 4 is a representation of an embodiment according to the present disclosure.

FIG. 5 is a representation of an embodiment according to the present disclosure.

FIG. 6 is a representation of an embodiment according to the present disclosure.

### DETAILED DESCRIPTION OF THE INVENTION

Embodiments of the invention described and claimed herein are not to be limited in scope by the specific embodiments herein disclosed, since these embodiments are intended as illustration of several aspects of the disclosure. Any equivalent embodiments are intended to be within the scope of this disclosure. Indeed, various modifications of the embodiments in addition to those shown and described herein will become apparent to those skilled in the art from the foregoing description. Such modifications are also intended to fall within the scope of the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

As used herein and in the appended claims, the singular forms “a,” “an,” and “the” include plural reference unless the context clearly dictates otherwise. Thus, for example, a reference to “a reactor” includes a plurality of reactors, such as in a series of reactors. In this document, the term “or” is used to refer to a nonexclusive or, such that “A or B” includes “A but not B,” “B but not A,” and “A and B,” unless otherwise indicated.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “cycloolefin” as used herein refers to the compound of formula I:

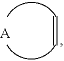

wherein A is a C₆-C₁₀ alkene chain with at least one carbon-carbon double bond. In one embodiment, A is a C₁₀ alkene with two double bonds. In another embodiment, A is a C₆ alkene with one double bond.

The term “ozonolysis transformation products” as used herein refers to the ozonolysis transformation product mixture of the compound of formula I comprising the compound of formula III:

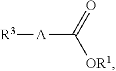

wherein A is a C₆-C₁₀ alkene chain with at least one double bond, R¹ is a C₁-C₁₀ alkyl, and R³ is an oxygen-containing functional group.

The term “alkene” as used herein refers to a linear or branched hydrocarbon olefin that has at least one carbon-carbon double bond.

The term “alkyl” or “alkylene” as used herein refers to a saturated hydrocarbon group which can be an acyclic or a cyclic group, and/or can be linear or branched unless otherwise specified.

The term “reagent” as used herein means a consumable material that provides the suitable R¹ functionality in the compound of formula IIa. In some embodiments, the reagent is polar. In other embodiments, the reagent provides a single continuous phase of the reaction. In yet another embodiment, the reagent improves the flowability characteristics of the reaction medium. In some embodiments, the reagent improves the heat transfer properties of the reaction medium.

The term “high purity”, as used herein, means at least about 90 wt. %, such as at least about 95 wt. %, such as at least about 96 wt. %, for example, 98 wt. % or higher.

The term “normal boiling point”, as used herein, means the boiling point of a component measured or estimated at 760 mmHg (1 atm.) pressure.

The term “low-boiling”, as used herein, means that the normal boiling point of the component(s) to which it refers is less than about 240° C.

The term “mid-boiling”, as used herein, means that the normal boiling point of the component(s) to which it refers is in the estimated range from about 240° C. to about 330° C.

All publications, including non-patent literature (e.g., scientific journal articles), patent application publications, and patents mentioned in this specification are incorporated by reference as if each were specifically and individually indicated to be incorporated by reference.

It is understood that the descriptions herein are intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” “third,” and the like are used merely as labels, and are not intended to impose numerical requirements on their objects.

One aspect of the disclosed process is directed to a method for separating a mixture comprising a compound of formula I:

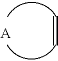

and a compound of formula III:

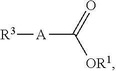

comprising distilling the mixture, wherein A is a C₆-C₁₀ alkene chain with at least one double bond, R¹ is a C₁-C₁₀ alkyl, and R³ is an oxygen-containing functional group.

Examples of the suitable distilling condition include, but are not limited to, sub-atmospheric, atmospheric or above-atmospheric pressures. In some embodiments, the pressure is maintained in the range from about 0.005 kPa to about 200.0 kPa. In other embodiments, the pressure is maintained in the range from about 0.01 kPa to about 100.0 kPa. In a further embodiment, the pressure is maintained in the range from about 0.02 kPa to about 50.0 kPa. In yet further embodiment, the pressure is maintained in the range from about 0.04 kPa to about 30.0 kPa. The pressure unit conversion of 1.0 kPa (kilo Pascals) equals 7.50 mmHg.

In some embodiments, the distillation temperature may be maintained in the range from about 30° C. to about 250° C. In other embodiments, the distillation temperature may be maintained in the range from about 40° C. to about 200° C. In a further embodiment, the distillation temperature may be maintained in the range from about 45° C. to about 175° C. In yet further embodiment, the distillation temperature may be maintained in the range from about 50° C. to about 150° C.

In one embodiment, the mixture is a crude mixture from ozonolysis of the compound of formula I.

### Ozonolysis and Transformation

One aspect of the '672 Publication is directed to a method of making a compound of formula

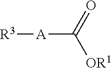

comprising:
a) contacting a compound of formula I:

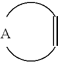

and a reagent with a medium comprising ozone to form a mixture comprising a compound of formula IIa:

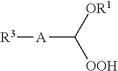

and the reagent;
b) exposing the mixture to a combination of temperature and pressure such that the reagent flashes to increase concentration of Formula IIa in the mixture without thermally degrading the compound of formula IIa component of the mixture;
c) contacting the concentrated mixture comprising the compound of formula IIa with an acid anhydride and a trialkyl amine; and
d) recovering a product comprising the compound of formula III;
   wherein A is a C₆-C₁₀ alkene chain with at least one double bond, R¹ is a C₁-C₁₀ alkyl, and R³ is an oxygen-containing functional group.

The compound of formula I may include cyclic trienes and cyclic dienes. Examples of the compound of formula I include, but are not limited to, cyclohexadiene, cycloheptadiene, cyclooctadiene, cyclooctatetraene, cyclododecadiene, cyclododectriene, cyclododecapentaene including isomers and mixtures thereof. In some embodiments, the compound of formula I is cyclododecatriene or cyclooctadiene. In a further embodiment, the compound of formula I is CDDT or COD. In another further embodiment, the compound of formula I is CDDT.

In some embodiments, the reagent is a C₁-C₁₀ alcohol. Examples of the suitable alcohol include, but are not limited to, methanol, ethanol, 1-propanol, 2-propanol, 1-butanol, 2-butanol, iso-butanol, t-butanol, and mixtures thereof. In some embodiments, the alcohol is 1-propanol, 2-propanol, 1-butanol, 2-butanol, iso-butanol, t-butanol, and mixtures thereof. In other embodiments, the reagent is a C₄-C₁₀ alcohol. Higher alcohols such as butanols, etc., are preferred.

In some embodiments, the reagent is anhydrous, preferably contains less than 0.5 wt. % water, or more preferably less than 0.1 wt. % water. In other embodiments, the water content may be no more than 0.08 wt %, preferably no more than 0.04 wt %.

In some embodiments, the amount of the reagent may vary and generally excess reagent may be used. In this context, the term “excess” is defined as the molar amount of the reagent that is more than the reacted compound of formula I. For purposes of the disclosed process, the molar ratio of the compound of formula I to the reagent may be about 100:1 to about 1:100, preferably about 25:1 to about 1:25, and more preferably about 10:1 to about 1:10. In one embodiment, the molar ratio of the compound of formula I to the reagent is about 4:1 to about 1:10. In another embodiment, the molar ratio of the compound of formula I to the reagent is about 6:1 to about 1:6. In yet another embodiment, the molar ratio of the compound of formula I to the reagent is about 3:1 to about 1:3.

In some embodiments, the ozonolysis reaction may be conducted in the presence of an optional inert solvent. In other embodiments, the inert solvent is a polar solvent. Examples of the suitable polar solvent include, but are not limited to, C₁-C₆ alkyl acetates, ethers, DMF, DMAc, DMSO, NMP, THF, and mixtures thereof.

In some embodiments, the conversion of the compound of formula I is from about 0% to about 100%. In a further embodiment, the conversion of the compound of formula I is from about 10% to about 95%. In other further embodiments, the conversion of the compound of formula I is from about 20% to about 90%, from about 30% to about 70%, or from about 30% to about 60%. In other embodiments, the conversion of the compound of formula I is at least 20%. In a further embodiment, the conversion is at least 25%.

As disclosed in the '360 Publication, the ozonolysis reaction can be conducted under conditions to selectively ozonize only one carbon-carbon double bond in the compound of formula I to form the compound of formula IIa. In a non-selective ozonolysis, more than one carbon-carbon double bonds are converted and non-selective products are formed. In some embodiments, the ozonolysis conditions favor the compound of formula IIa with the preservation of “A” as in the compound of formula I. For example, in the compound of formula IIa from the ozonolysis of CDDT, “A” should be a C₁₀ alkene chain with two carbon-carbon double bonds. Due to the cleaving of more than one double bonds, in some embodiments, the non-selective products contain the compound of formula IIa with fewer carbon numbers in “A” than the compound of formula I.

In some embodiments, the ozonolysis effluent may comprise from about 0 wt. % to about 50 wt. % the compound of formula I, from about 0 wt. % to about 80 wt. % reagent, from about 0 wt. % to about 50 wt % the compound of formula IIa, and up to about 15 wt. % non-selective products. The non-selective products may include compounds having two terminal oxygenated groups, which include dialdehydes, diacids, diesters, acid-esters, aldehyde-acids. In some embodiments, at least some of the non-selective products are saturated, for example, linear C₄ species. In a preferred embodiment, the ozonolysis effluent comprises from about 0 wt. % to 50 wt. % of the compound of formula I, from about 0 wt. % to about 80 wt. % reagent, from about 0 wt. % to about 50 wt. % the compound of formula IIa, and up to about 10 wt. % non-selective products. In some embodiments, the ozonolysis effluent is a stable, flowable liquid at ambient conditions.

In some embodiments, the compound of formula IIa is formed with a selectivity of at least 50%. In other embodiments, the compound of formula IIa is formed with a selectivity of at least 60%. In another embodiment, the compound of formula IIa is formed with a selectivity of at least 70%. In other embodiments, the selectivity for the compound of formula IIa is at least 80%. In another embodiment, the selectivity for the compound of formula IIa is at least 85%. In a further embodiment, the selectivity for the compound of formula IIa is at least 90%. In another further embodiment, the selectivity for the compound of formula IIa is at least 95%. In some embodiments, the selectivity for the non-selective products is less than 10%. In a further embodiment, the selectivity for the non-selective products is less than 5%.

In some embodiments, the ozonolysis reaction may be conducted at a temperature of less than 50° C., preferably from about −25° C. to about 50° C., more preferably from about 0° C. to about 40° C., and most preferably from about 0° C. to about 25° C. The ozonolysis reaction is exothermic and, in some embodiments, the temperature of the reactor is maintained by a cooling system, such as an active jacketed cooler.

In some embodiments, the ozonolysis reaction may be conducted at a pressure from about 100 torr to about 200 Psig. In other embodiments, the ozonolysis reaction may be conducted at a pressure from about 100 torr to about 100 Psig. In a further embodiment, the ozonolysis reaction may be conducted at a pressure from about 0 Psig to about 50 Psig, preferably from about 0 Psig to about 25 Psig, more preferably from about 0 Psig to about 20 Psig, and most preferably from about 0 Psig to about 10 Psig. In some embodiments, the vacuum operation may be most suitable for removing the reaction heat via evaporative cooling and so long as the reaction performance is not adversely impacted.

In one embodiment, the compound of formula IIa is formed from ozonolysis of CDDT, wherein A is —CH₂—CH₂—CH═CH—CH₂—CH₂—CH—CH—CH₂—CH₂—. This compound of formula IIa is thermally stable.

In some embodiments, after the excess reagent is removed, the enriched product stream can be catalytically transformed in the presence of a catalyst to form the compound of formula III:

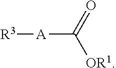

In other embodiments, a homogeneous catalyst complex was used for the catalytic transformation reaction. In yet another embodiment, a heterogeneous catalyst may be used for the transformation reaction.

The catalytic transformation is selective to the peroxy bond and does not react with the double bonds of a compound of formula II:

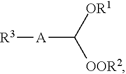

wherein A is a C₆-C₁₀ alkene chain with at least one double bond; R¹ is a C₁-C₁₀ alkyl; R² is H or acetyl; and R³ is an oxygen-containing functional group.

In some embodiments, the anhydride and amine-acid complex are added to a mixture containing the compound of formula IIa that is substantially free of the reagent. In another embodiment, the reagent in the mixture is less than 1 wt %. In yet another embodiment, the reagent in the mixture is less than 0.5 wt %.

In some embodiments, the conversion for catalytic transformation from the compound of formula IIa to the compound of formula III is between 0 and 100%. In one embodiment, the conversion is in the range of about 0 to about 20%, about 20 to about 40%, about 40% to about 60%, about 60% to about 80%, about 80% to about 100%. In another embodiment, the conversion is at least 90%, preferably at least 95%, and more preferably at least 99%. The catalytic transformation may be conducted at temperatures less than 50° C., preferably range from about 0° C. to about 50° C., and more preferably from about 5° C. to about 40° C.

The catalytic transformation is exothermic and, in some embodiments, the temperature of the reactor is maintained by a cooling system, such as an active jacketed cooler, to maintain a temperature of less than, e.g., 40° C. In some embodiments, the catalytic transformation may be conducted at a pressure from about 0 Psig to about 30 Psig. In other embodiments, the vacuum condition may be suitable when evaporative cooling is used. In a further embodiment, the catalytic transformation may be conducted at a pressure from about 0 Psig to about 5 Psig.

In some embodiments, the catalytic complex is an azeotropic acid-amine complex. This catalytic complex may be recovered from the product mixture via azeotropic distillation and recycled back to the transformation reactor.

In one embodiment, the compound of formula III in the mixture is from about 0.1% to about 99% by weight. In another embodiment, the compound of formula III in the mixture is from about 5% to about 90% by weight. In yet another embodiment, the compound of formula III in the mixture is from about 10% to about 85% by weight. In a further embodiment, the compound of formula III in the mixture is from about 15% to about 80% by weight. In another further embodiment, the compound of formula III in the mixture is from about 20% to about 50% by weight.

In one embodiment, the compound of formula I in the mixture is from about 1% to about 99% by weight. In another embodiment, the compound of formula I in the mixture is from about 10% to about 95% by weight. In yet another embodiment, the compound of formula I in the mixture is from about 15% to about 90% by weight. In a further embodiment, the compound of formula I in the mixture is from about 20% to about 85% by weight. In yet another further embodiment, the compound of formula I in the mixture is from about 20% to about 50% by weight.

In some embodiments, R¹ is a C₁-C₆ alkyl. In one embodiment, R¹ is a C₁-C₄ alkyl. In another embodiment, R¹ is a C₂-C₄ alkyl. In a further embodiment, R¹ is propyl or butyl.

In some embodiments, A is a C₆ or C₁₀ alkene chain with at least one double bond. In one embodiment, A is a C₁₀ alkene with two double bonds. In another embodiment, A is a C₆ alkene with one double bond.

In some embodiments, R³ is an aldehyde, an acid, or an ester group. In a further embodiment, R³ is an aldehyde or an acid group. In another further embodiment, R³ is an aldehyde group.

In one embodiment, the mixture further comprises an acid. Examples of suitable acids include, but are not limited to, acetic acid, succinic acid, maleic acid. In a further embodiment, the acid is acetic acid.

In one embodiment, the acetic acid in the mixture is from about 0.1% to about 99% by weight. In another embodiment, the acetic acid in the mixture is from about 5% to about 90% by weight. In yet another embodiment, the acetic acid in the mixture is from about 10% to about 85% by weight. In a further embodiment, the acetic acid in the mixture is from about 15% to about 80% by weight. In yet another further embodiment, the acetic acid in the mixture is from about 20% to about 50% by weight.

In one embodiment, the mixture is distilled through at least one flash distillation. In another embodiment, the flash distillation is a short path distillation.

In a further embodiment, the at least one flash distillation is a single-stage flash distillation. See, e.g., Examples 2-17.

In another embodiment, the at least one flash distillation is a two-stage flash distillation. See, e.g., Example 18. In a further embodiment, the first flash distillation is to remove an overhead comprising the acid and the compound of formula I from the mixture.

In some embodiment, the overhead comprises about 0.1% to about 90% of the acid. In a further embodiment, the overhead comprises about 5% to about 80% of the acid. In yet another further embodiment, the overhead comprises about 10% to about 75% of the acid.

In other embodiments, the acid and the compound of formula I in the overhead are separable by liquid-liquid phase separation.

In another embodiment, the second flash distillation is to further remove the compound of formula I from the mixture.

In some embodiments, the bottom stream after the second flash distillation contains more than 70% of the compound of formula III. In a further embodiment, the bottom stream after the second flash distillation contains more than 75%, 80%, 85%, 90% or 95% of the compound of formula III.

In one embodiment, the mixture further comprises an acid anhydride and an amine. Examples of the acid anhydride include, but are not limited to, acetic anhydride, succinic anhydride, maleic anhydride, other anhydrides belonging to the general anhydride family and mixtures thereof. In a further embodiment, the acid anhydride is acetic anhydride. Examples of the amine include, but are not limited to, triethyl amine, diethanol amine, tributyl amine, pyridine, other unsubstituted or substituted amities belonging to the general amines family and mixtures thereof. In an embodiment, the amine is triethyl amine.

The distillative units for use in the disclosed process may comprise either packing or trays to provide mass transfer, however, those columns in which two liquid phases are present may preferentially use trays to ensure better liquid mixing.

In some embodiments, the liquid-liquid phase separation may be performed in a phase separator device that may comprise simple decanters and may also comprise coalescers. Phase separators may be either horizontally or vertically oriented and must be designed with sufficient residence time and cross-sectional area to enable adequate time for phase separation to occur. A coalescer can advantageously be used to enhance the coalescence of droplets to facilitate phase separation. The coalesces may consist of wire-wool, corrugated sheets or other such common designs for such devices.

An embodiment of the feed mixture to be separated by the process of the present invention may comprise, by weight, from about 0% to 5%, e.g., 0.05% to 2% water. Other extraneous components of the mixture, if any, will be small amounts of other organic species, such as those having or providing undesirable impurities.

In some embodiments, the at least one flash distillation is a three-stage flash distillation. See, e.g., Example 31. In a further embodiment, the third-stage flash distillation is to reject a bottom stream comprising high-boiling impurities and to recover a refined overhead stream of the compound of formula III from the mixture.

In some embodiments, the bottom stream after the second flash distillation containing more than 70% of the compound of formula III is fed to the third-stage flash distillation. In other embodiments, the third-stage flash distillation is to further refine the compound of formula III from the mixture.

In some embodiment, the overhead stream after the third-stage flash distillation contains more than 70% of the compound of formula III. In a further embodiment, the overhead stream after the third-stage flash distillation contains more than 75%, 80%, 85%, 90% or 95% of the compound of formula III.

The third-stage flash distillative unit for use in the disclosed process may comprise either packing or trays to provide mass transfer, however, those columns in which two liquid phases are present may preferentially use trays to ensure better liquid mixing.

An embodiment of the feed mixture to be separated by the multi-stage flash distillative process of the present invention may comprise, by weight, from about 0% to 5%, e.g., 0.05% to 2% water. Other extraneous components of the mixture, if any, will be small amounts of other organic species, such as those having or providing undesirable impurities.

In an embodiment, the disclosed process may apply to recover the acid from other similar reaction mixtures comprising the acid.

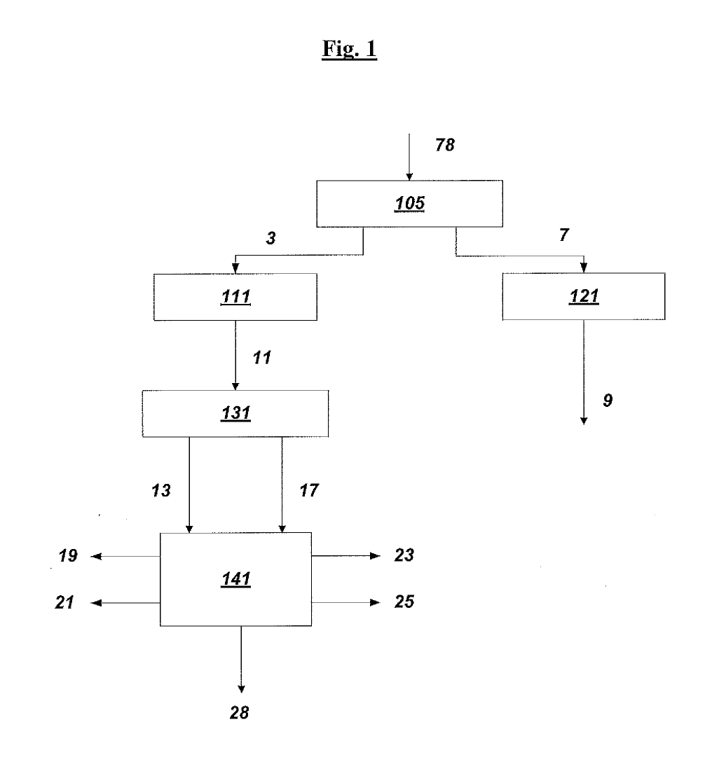

### Overview of FIG. 1

A more detailed description of a representative process for the multi-component mixture separation is shown in FIG. 1, which provides a simplified schematic representation of such a process. FIG. 1 shows a first distillative unit 105, where a mixture, comprising an anhydride, alkyl amine, acid, the compound of formula I and its ozonolysis transformation products, and optionally, other organic species providing undesirable impurities, is fed via stream 78.

In an embodiment, the first distillative unit 105 may be sufficient to provide the short residence time for the feed. In another embodiment, the first distillative unit 105 may be of a thin liquid film type. In a further embodiment, the first distillative unit 105 may be from a class of short-residence time distillation devices, including but not limited to, thin-film evaporator, falling-film evaporator, wiped-film evaporator, short-path distillation, circulating-film evaporator and flash evaporator. In yet another embodiment, the first distillative unit 105 may be a short-path distillation column.

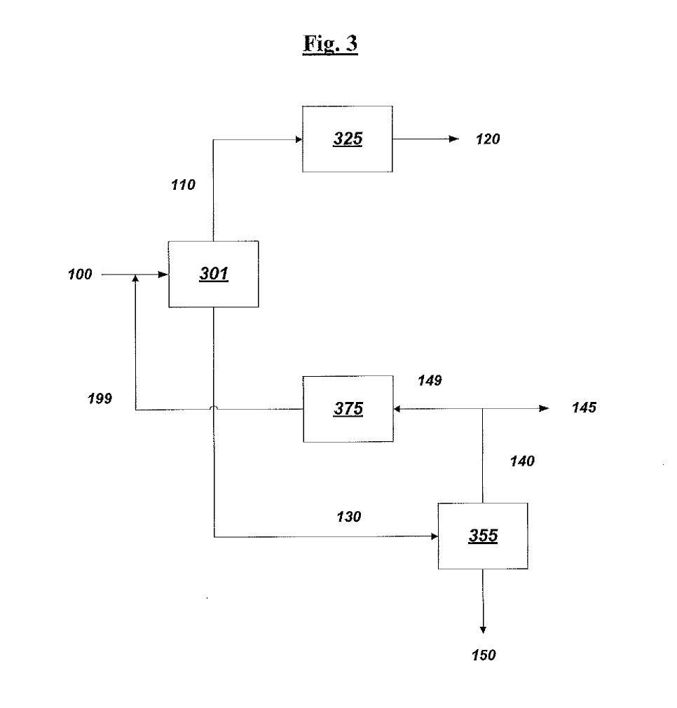

In some embodiments, the first distillative unit 105 may be operated in a single-stage mode. In other embodiments, the first distillative unit 105 may be a sequential multi-stage arrangement with intra-stage or inter-stage recycles as represented in FIG. 3. By intra-stage recycle, applicants mean recycle of any one or all of unit output streams back to the feed to the same unit stage. By inter-stage recycle, applicants mean recycle of any or all of unit output streams back to the feed to another unit stage.

In an embodiment, the first distillative unit 105 may be equipped with adequate internal heat exchange surfaces for supplying heat to the material and condensing the vapors into the top fraction stream. In another embodiment, the first distillative unit 105 may be equipped with adequate external heat exchange surfaces for supplying heat to the material and condensing the vapors into the top fraction stream.

The temperature and pressure environment inside the first distillative unit 105 may be controlled and maintained such that the mixture feed via stream 78 is rapidly separated into a top fraction stream 3 and a bottom fraction stream 7.

In some embodiments, the temperature inside unit 105 may be equilibrated in the range from about 30° C. to about 200° C. In other embodiments, the temperature inside unit 105 may be equilibrated in the range from about 40° C. to about 175° C. In a further embodiment, the temperature inside unit 105 may be equilibrated in the range from about 45° C. to about 165° C. In yet further embodiment, the temperature inside unit 105 may be equilibrated in the range from about 50° C. to about 160° C.

The residence time in unit 105 is dependent on the composition of stream 78 and the pressure-temperature conditions employed in unit 105. In some embodiments, the residence time of the liquid phase inside unit 105 may be attained in the range from about 0.5 second to about 60 minutes. In other embodiments, the residence time of the liquid phase inside unit 105 may be in the range from about 1 second to about 45 minutes. In a further embodiment, the residence time of the liquid phase inside unit 105 may be in the range from about 5 seconds to about 35 minutes. In yet further embodiment, the residence time of the liquid phase inside unit 105 may be in the range from about 10 seconds to about 30 minutes.

In some embodiments, the pressure inside unit 105 may be equilibrated in the range from about 0.005 kPa to about 200.0 kPa. In other embodiments, the pressure inside unit 105 may be equilibrated in the range from about 0.01 kPa to about 100.0 kPa. In a further embodiment, the pressure inside unit 105 may be equilibrated in the range from about 0.02 kPa to about 50.0 kPa. In yet further embodiment, the pressure inside unit 105 may be equilibrated in the range from about 0.04 kPa to about 30.0 kPa. The pressure unit conversion of 1.0 kPa (kilo Pascals) equals 7.50 mmHg.

In some embodiments, the effective heat transfer surface area inside unit 105 may be in the range from about 0.0001 m² per unit g/min feed rate to about 1.0 m² per unit g/min feed rate. In other embodiments, the effective heat transfer surface area inside unit 105 may be in the range from about 0.0003 m² per unit g/min feed rate to about 0.5 m² per unit g/min feed rate. In another embodiment, the effective heat transfer surface area inside unit 105 may be in the range from about 0.0005 m² per unit g/min feed rate to about 0.1 m² per unit g/min feed rate.

Now referring to FIG. 1, the distillative unit 105, described herein, obtains a top fraction stream 3 and a bottom fraction stream 7. The top fraction stream 3 feeds to a total condenser unit 111 operating in the temperature range of about 5° C. to about 15° C. and the equilibrated pressure as that of the top pressure of the distillative unit 105. There is no such restriction on what condensation temperature to use so long as at least part of the material is flowable and processeble. The condensed liquid stream 11 is drawn off from the total condenser 111 and flowed to a first phase separator 131.

The first phase separator 131 may be sub-cooled by chilled brine, water-cooled or maintained at room temperature (e.g., 20° C.). The pressure inside the first phase separator 131 may be either reduced, atmospheric or above atmospheric. The first phase separator 131 provides sufficient residence time and cross-sectional area for stream 11 to spontaneously separate into two liquid phases; a top phase stream 13, and a bottom phase stream 17. In one embodiment, the two phases have a well-defined phase interface separating the two.

In some embodiments, either of the two phases in the first phase separator 131 may be separated by any such industrial methods as, but not limited to, gravity decantation, overflow weir, bottom phase pump-out and such. In other embodiments, the density difference between the two separated phases may be adequate to draw individual phases out of the first phase separator 131 while monitoring the phase interface.

Optionally, a second phase separator (not shown) may be employed downstream of the first phase separator 131 in series or parallel for either stream 13 or 17. In some embodiments, the second phase separator may provide the extra residence time and cross-sectional area for the two individual streams if the first phase separator 131 is only partially effective.

In some embodiments, the top phase-to-bottom phase flow split fraction of the feed entering the first phase separator 131 may be in the range from 0.01 to 0.99 (wt/wt). In other embodiments, the top phase-to-bottom phase flow split fraction of the feed entering the first phase separator 131 may be in the range from 0.05 to 0.95 (wt/wt). In one embodiment, the top phase-to-bottom phase flow split fraction of the feed entering the first phase separator 131 is in the range from 0.1 to 0.9 (wt/wt), preferably in the range from 0.1 to 0.89 (wt/wt), more preferably in the range from 0.1 to 0.88 (wt/wt).

In some embodiments, stream 13 may comprise substantial components that have lower polarity relative to the stream 17. In other embodiments, stream 13 may comprise majority of the compound of formula I present in stream 78. In one embodiment, stream 17 is mostly the acid, anhydride and amine. In another embodiment, stream 17 is a concentrated mixture of anhydride, acid, and amine. In yet another embodiment, stream 17 is a mixture of acid and amine.

Referring to FIG. 1, the separated streams 13 and 17 may enter a second distillative unit 141 for further component separation. In some embodiments, the two streams may be sequentially processed in the second distillative unit 141. In other embodiments, only stream 13 may be processed in the second distillative unit 141 and stream 17 may be circulated back (not shown) to any of the first distillative unit 105 or the phase separator unit 131. In another embodiment, only stream 17 may be processed in the second distillative unit 141 and stream 13 may be circulated back (not shown) to any of the first distillative unit 105 or the phase separator unit 131.

The second distillative unit 141 may comprise at least one separation unit with refluxing, boil-up, side draw and pump-around capabilities. In one embodiment, stream 13 may be fractionated to obtain the high-purity compound of formula I via stream 28 and a mid-boiling component stream 25. Depending on the composition and constituents present, stream 25 may either undergo further refinement for useful component recovery (not shown) or discarded.

In some embodiments, stream 17 may be fractionated in the second distillative unit 141 to obtain about 90 to about 100 wt. % acid via stream 23, a stream comprising anhydride, acid and amine via stream 21, and a low-boiling component stream 19. Depending on the composition and constituents present, stream 19 may either undergo further refinement for useful component recovery (not shown) or discarded.

The bottom fraction stream 7, obtained from the first distillative unit 105, is taken to a cooler unit 121, wherein stream 7 is cooled to room temperature (20° C.). The cooled stream 9 is a stable, flowable liquid that could be pumped to a storage facility. In one embodiment, the obtained stream 9 may be stored under the nitrogen atmosphere in the temperature range of between −10° C. and 25° C.

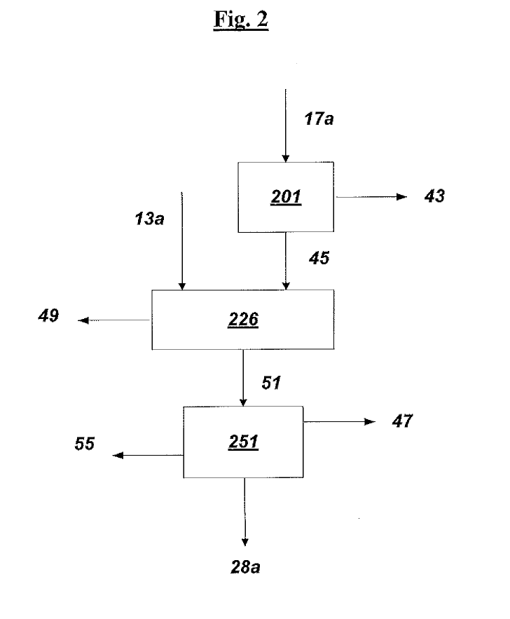

### Overview of FIG. 2

FIG. 2 shows a non-limiting embodiment of distillative unit sequence, which may be used as separation unit 141, shown in FIG. 1. In FIG. 2, stream 13a represents the top phase stream 13 (FIG. 1) that is obtained from the first phase separator 131 or equivalent. In FIG. 2, stream 17a represents the bottom phase stream 17 (FIG. 1) that is obtained from the first phase separator 131 or equivalent.

Stream 17a is fed to a second distillative unit 201 which comprises of at least twenty-nine theoretical stages, refluxing and boil-up capabilities. The second distillative unit 201 may be operated under the pressure of 20-40 kPa (head)/30-75 kPa (base) and in the temperature range of about 45° C. at the column head to about 130° C. at the column base. The column feed enters in the lower half section of the unit 201, preferably at the lower third of the column, more preferably at the ¾^{th} of the column length measured from the top. There are about 75% of total theoretical stages above the feed entry location and about 25% of the total stages below the feed entry location for the unit 201.

The acid present in stream 17a is concentrated in the overhead vapors, condensed below its dew point and drawn as stream 43. Upon stripping of the acid in the overhead, the remaining material is concentrated at the base and drawn as liquid stream 45. Stream 45 comprises the anhydride, amine-acid complex and components having the normal boiling point higher than the acid.

In some embodiments, the conditions inside the second distillative unit 201 are maintained such that stream 43 is comprised of, by weight, >90% acetic acid, <5% anhydride and <5% C4 compounds. In other embodiments, stream 43 is a high purity, e.g., 96% acetic acid with <1% anhydride and <5% C4 compounds. In one embodiment, stream 45 drawn from the second distillation column 201 contains a 70:30 (wt/wt) complex of acid:amine.

In some embodiments, the acid-depleted stream 45 may be combined with the stream 13a and the combined stream may be fed to a third distillative unit 226. In other embodiments, the combined feed to the third distillative unit 226 may comprise 0-80 wt % compound of formula I, 0-50 wt % anhydride, 0-25 wt % amine, 0-25 wt % acid.

The third distillative unit 226 comprises minimum twenty separation stages, refluxing and boil-up capabilities, and is maintained at the reduced pressure in the range of 0.1 to 75 kPa and the temperature range of about 45° C. at the column head to about 200° C. at the column base. In some embodiments, the pressure in the third distillative unit 226 may be maintained in the range of 0.5 to 50 kPa, preferably in the range of 1.0 to 30 kPa, more preferably in the range of 1.5 to 20 kPa.

In some embodiments, the feed to the third distillative unit 226 may enter at the mid column length. In other embodiments, there may be about 50% of total theoretical stages above the feed entry and about 50% of total stages below the feed entry for the unit 226.

In some embodiments, the third distillative unit 226 comprises one or multiple packing sections, such as BX packing with 11″ HETP [Height Equivalent Theoretical Plate], to obtain the required separation. In other embodiments, the third distillative unit 226 comprises two individual packing sections, such as BX packing with 11″ HETP, to obtain the first separation stage section above the feed entry and the second stripping stage section below the feed entry.

In one embodiment, stream 49 drawn from the third distillative unit 226 may comprise, by weight, between 0% to 50% anhydride, between 0% to 70% acid, between 0% to 50% amine, between 0% to 25% C₄ organic compounds and <5% compound of formula I. In another embodiment, stream 49 drawn from the third distillative unit 226 may comprise between 5% to 45% anhydride, between 10% to 60% acid, between 5% to 30% amine, between 0% to 10% C₄ organic species and less than 1% of the compound of formula I.

In some embodiments, the molar ratio of acid and amine present in stream 49 may range from about 1:100 to about 100:1. In one embodiment, the molar ratio of acid and amine in stream 49 may range from about 1:25 to about 25:1. In another embodiment, the molar ratio of acid and amine in stream 49 may range from about 1:10 to about 10:1. In a further embodiment, the molar ratio of acid and amine in stream 49 may range from about 1:6 to about 6:1. In yet another embodiment, the molar ratio of acid and amine in stream 49 is about 1:1.

In some embodiments, stream 49 may contain, by weight, between 0% and 999% anhydride. In other embodiments, stream 49 may contain between 1% and 60% anhydride. In another embodiment, stream 49 may contain between 5% and 50% anhydride. In yet another embodiment, stream 49 may contain between 10% and 45% anhydride.

The other components present in stream 45 are concentrated at the base of the third distillative unit 226 and drawn out as liquid stream 51. Stream 51, substantially free of anhydride, amine, acid and/or the amine-acid complex, is fed to a fourth distillative unit 251. In an embodiment, stream 51 may comprise 50-95 wt % compound of formula I. In another embodiment, stream 51 may comprise 60-90 wt % compound of formula I.

In some embodiments, the fourth distillative unit 251 may comprise of a minimum of twelve theoretical stages, refluxing, boil-up and side draw capabilities. The feed, i.e., stream 51, may enter at about 30% of the column length measured from the top, which yields about three to four theoretical stages above the feed location and about 70% of total stages below the feed location. A provision is made, via a liquid collection tray, to take a liquid side draw from about ⅔^{rds} of the column length measured from the top, which may be a mid-point location between the feed tray and the column bottom. In other embodiments, there may be about 30% of total theoretical stages above the feed distributor, about 35% of total theoretical stages between the feed distributor and liquid side draw tray and about 35% of total theoretical stages below the side draw tray.

The fourth distillative unit 251 operates under the reduced pressure condition, preferably less than 50 kPa, and in the temperature range of about 35° C. at the column head to about 150° C. at the column base. The compound of formula I may concentrate in the mid-section between the feed tray and the column bottom, wherein a liquid side-draw is taken as stream 28a. Stream 28a comprises the compound of formula I in high purity which is substantially free of the anhydride, acid or amine.

In some embodiments, stream 28a may comprise, by weight, about 75-99.9% compound of formula I and about 0.1-25% mid-boiling components present in the feed stream 78 [in FIG. 1]. In other embodiments, stream 28a may comprise about 75-95% compound of formula I and about 5-25% mid-boiling components.

In some embodiments, the low-boiling components are taken out at the column top as stream 47. Stream 47 may be substantially concentrated in the low-boiling components, such as but not limited to C₁-C₄ organic species, e.g., C₄ dialdehyde. In other embodiments, the mid-boiling components may be concentrated at the column base and taken out as stream 55. In an embodiment, stream 55 may be substantially concentrated in the mid-boiling components, such as about 50% to about 60%, about 60 to about 70%, about 70% to about 80%, about 80% to about 90%, about 90% to about 100%, and combinations therebetween.

In some embodiments, the fourth distillative unit 251 may be a divided wall distillative column; wherein the vapor-liquid traffic may be strategically distributed in more than one axially partitioned section of the column and the temperature-pressure environment maintained in each axially partitioned section such as to obtain the desired separation.

In some embodiments, the fourth distillative unit 251 may consist of a main column with a short side concentrator column (not shown); wherein either a vapor or liquid draw may be taken from the main column location that is substantially concentrated in the compound of formula I, further concentrating the side draw in the side concentrator column, recovering the compound of formula I in the side concentrator column overhead, and returning the side concentrator bottoms liquid, that is devoid of the compound of formula I, back to the main column. In an embodiment, the side concentrator column that is serving to the main column may be a simple flash separator/condenser. In another embodiment, the side concentrator column may be integrated via a pump-around loop at the desired tray location that is substantially rich in the compound of formula I.

### Overview of FIG. 3

FIG. 3 shows a non-limiting embodiment of distillative unit sequence, which may be used as first distillative unit 105, shown in FIG. 1. In FIG. 3, stream 100 represents the feed stream 78 (FIG. 1). In FIG. 3, stream 150 represents the bottom fraction stream 7 (FIG. 1). In FIG. 3, stream 120 represents the top fraction stream 3 (FIG. 1).

In some embodiments, the first distillative unit 105 (FIG. 1) may be a sequential multi-stage arrangement with intra-stage or inter-stage recycles. In other embodiments, the first distillative unit 105 (FIG. 1) may be a sequential two-stage arrangement with inter-stage recycle, as represented by a first stage unit 301 (FIG. 3), a second stage unit 355 (FIG. 3) and a recycle stream 199 (FIG. 3).

Referring specifically to FIG. 3, the temperature and pressure environment inside the first stage unit 301 may be controlled and maintained such that the mixture feed via stream 100 is separated into a top fraction stream 110 and a bottom fraction stream 130.

In some embodiments, the temperature inside the first stage unit 301 may be equilibrated in the range from about 30° C. to about 200° C. In other embodiments, the temperature inside the first stage unit 301 may be equilibrated in the range from about 40° C. to about 175° C. In a further embodiment, the temperature inside the first stage unit 301 may be equilibrated in the range from about 45° C. to about 165° C. In yet further embodiment, the temperature inside the first stage unit 301 may be equilibrated in the range from about 50° C. to about 160° C.

In some embodiments, the pressure inside the first stage unit 301 may be equilibrated in the range from about 0.005 kPa to about 200.0 kPa. In other embodiments, the pressure inside the first stage unit 301 may be equilibrated in the range from about 0.01 kPa to about 100.0 kPa. In a further embodiment, the pressure inside the first stage unit 301 may be equilibrated in the range from about 0.02 kPa to about 50.0 kPa. In yet further embodiment, the pressure inside the first stage unit 301 may be equilibrated in the range from about 0.04 kPa to about 30 kPa. The pressure unit conversion of 1.0 kPa (kilo Pascals) equals 7.50 mmHg.

In an embodiment, the bottom fraction stream 130 may be fed to the second stage unit 355, while the top fraction stream 110 may be fed to a first-stage condenser unit 325. In one embodiment, the first-stage condenser unit 325 may be operated in the sub-cooled temperature region, preferably in the −10° C. to 15° C., more preferably in the −5° C. to 10° C. range. The condensed sub-cooled liquid stream 120 is drawn out of the first-stage condenser unit 325 by the appropriate means.

In some embodiments, the second stage unit 355 may be maintained at the temperature and pressure environment such that the feed stream 130 is separated into a top fraction stream 140 and a bottom fraction stream 150.

In some embodiments, the temperature inside the second stage unit 355 may be equilibrated in the range from about 30° C. to about 200° C. In other embodiments, the temperature inside the second stage unit 355 may be equilibrated in the range from about 40° C. to about 175° C. In a further embodiment, the temperature inside the second stage unit 355 may be equilibrated in the range from about 45° C. to about 165° C. In yet further embodiment, the temperature inside the second stage unit 355 may be equilibrated in the range from about 50° C. to about 160° C.

In some embodiments, the pressure inside the second stage unit 355 may be equilibrated in the range from about 0.005 kPa to about 200.0 kPa. In other embodiments, the pressure inside the second stage unit 355 may be equilibrated in the range from about 0.01 kPa to about 100.0 kPa. In a further embodiment, the pressure inside the second stage unit 355 may be equilibrated in the range from about 0.02 kPa to about 50.0 kPa. In yet further embodiment, the pressure inside the second stage unit 355 may be equilibrated in the range from about 0.03 kPa to about 30.0 kPa.

In some embodiments, the top fraction stream 140 may be routed to a second-stage condenser unit 375 via stream 149. In other embodiments, the top fraction stream 140 may be either partially or totally drawn out via stream 145.

In other embodiments, the second-stage condenser unit 375 may be operated in the sub-cooled temperature region, preferably in the −10° C. to 15° C., more preferably in the −5° C. to 10° C. range. The condensed sub-cooled liquid stream 199 is drawn out of the second-stage condenser unit 375 by the appropriate means.

In an embodiment, the stream 199 in entirety may be combined with the mixture feed stream 100 and the combined stream may be fed to the first-stage unit 301. In another embodiment, a portion of the stream 199 may be taken out (not shown) of the recycle arrangement by the suitable means.

In some embodiments, the second-stage unit 355 in combination with the recycle stream 199 may be used to enrich the low-boiling components of the feed stream 100 into the top fraction stream 110. In other embodiments, the second-stage unit 355 may be used to purge out the accumulated impurities via the stream 145, either intermittently or continuously.

Another aspect of the disclosed process is directed to a method of separating a miscible mixture comprising the compound of formula I:

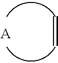

and the acid into two phases, comprising adding a phase-separation agent to the mixture, wherein A is a C₆-C₁₀ alkene chain with at least one double bond.

In some embodiments, the acid is acetic acid.

Examples of the suitable phase-separation agent include, but are not limited to, amines, water, nitriles, hydrocarbons, and mixtures thereof.

In some embodiments, the phase-separation agent is an amine or water. Examples of the amine include, but are not limited to, primary amines, secondary amines, tertiary amines, heterocyclic amines, and mixtures thereof. The primary amines may include alkyl amines such as isopropyl amine. The secondary amines may include diisopropyl amine, diethanol amine. The tertiary amines may include trialkyl amines such as triethyl amine, tributyl amine. The heterocyclic amines may include pyridine. In other embodiments, the amine can be in a free base, a salt or a complex form.

In some embodiments, water can be de-ionized (“DI”) water, high-purity water, process condensate water, boiler-feed water, salt water, water obtained from a water-wash process, brine, an aqueous amine solution or a water-based emulsion with sufficient amount of water to accomplish the phase separation. In other embodiments, sufficient water for the phase separation may be provided by a wet, non-reactive organic solvent. Whatever the source may be, the water stream used shall not contain impurities that are undesired to the current process.

In some embodiments, no more than 50% by weight of the phase separation agent is added to the mixture. In a further embodiments, no more than 40% by weight of the phase separation agent is added to the mixture. In another further embodiments, no more than 35% by weight of the phase separation agent is added to the mixture. In a further embodiments, no more than 30% by weight of the phase separation agent is added to the mixture. In another further embodiments, no more than 25% by weight of the phase separation agent is added to the mixture.

In some embodiments, the phase separation agent that is added to the mixture is between about 0.1% and about 50% by weight. In other embodiments, the phase separation agent that is added to the mixture is between about 0.2% and about 40% by weight. In a further embodiment, the phase separation agent that is added to the mixture is between about 0.3% and about 35% by weight. In another further embodiment, the phase separation agent that is added to the mixture is between about 0.4% and about 30% by weight. In yet another further embodiment, the phase separation agent that is added to the mixture is between about 0.5% and about 25% by weight.

The following Examples demonstrate the present invention and its capability for use. The invention is capable of other and different embodiments, and its several details are capable of modifications in various apparent respects, without departing from the scope and spirit of the present invention. Accordingly, the Examples are to be regarded as illustrative in nature and not as restrictive. Likewise, the below Examples illustrate non-limiting modes of carrying out the disclosed process with the particular arrangement of the units as described above. All percentages are by weight unless otherwise indicated.

In Examples, Tables and Figures of the present disclosure, “HOAc” means acetic acid component; “ACAN” means acetic anhydride component; “TEA” means triethylamine component; “HOAc:TEA Complex” or “acid:amine complex” or simply “complex” mean acetic acid:triethylamine complex; and “BuOAc” means butyl acetate component.

### Example 1

### Preparation of Mixture Comprising a Compound of Formula I, Compound of Formula III, Acid and Other Organic Species

A 500 ml jacketed round-bottom flask is fitted with a dry ice condenser, mechanical stirrer, stainless steel feed tube for sub-surface ozone gas addition and a fourth port for addition of reagents, sampling and thermocouple connection. A dry gas mixture containing 21% oxygen in argon is fed to an ozone generator (Pacific Ozone). The exit gas from the ozone generator is flowed through an ozone monitor (Teledyne Instruments) for 30 min to observe stable ozone concentration in the feed gas. The reaction temperature is maintained at a desired target via jacketed cooling. The exit gas containing residual oxygen and argon are passed through a dry ice cold trap to recover any low-boiling components. Upon reaction time completion, dry nitrogen is passed into the reactor for 30 min to displace any residual ozone and oxygen and the vessel is warmed to room temperature. 1,5,9-cyclododecatriene (CDDT) is used as received from INVISTA™ Specialty Intermediates. Table 1 depicts a typical composition.

TABLE 1
Typical Composition, wt %

| | |
|---|---|
| 1,5,9-Cyclododecatriene | >99 |
| Tert-butyl catechol | 30-50 ppm |
| Isomers | |
| cis, trans, trans | 98 |
| trans, trans, trans | 1.5 |
| cis, cis, trans | 0.3 |
| cis, cis, cis | 0.1 |

A steady concentration of 26.3 g ozone/m³ in 3 liter/min argon flow is sparged into the reaction vessel for 114 minutes containing 60 g (0.370 moles) of CDDT and 27.4 g of fresh, dry n-butyl alcohol. The reaction is carried out at 5.0° C. bulk temperature. When the reaction is complete, excess alcohol is flashed off at 50° C. and under vacuum. To the concentrated reaction intermediate a cooled liquid mixture of 35.2 g acetic anhydride and 7.5 g triethylamine is added via pump at an average feed rate of 2.67 g/min with 650 RPM stirring of the reaction mixture. The reaction exotherms, observed during these additions, are managed by active jacketed cooling to ensure the temperature is maintained at or below 40° C. The reaction mixture is allowed to reach room temperature and stirred for additional 30 minutes for completion. 112 g of one-phase liquid reaction product is recovered. The final GC analysis indicates 47.7% CDDT conversion. The normalized molar selectivity of the reacted CDDT is 90.2% for n-butyl ester of 12-oxo-dodeca-4,8-dieneoic acid (i.e., the compound of formula III), 1.6% for Dodeca-4,8-diene-1,12-dialdehyde, 2.0% for 12-oxo-dodeca-4,8-dieneoic acid, 3.5% for combined C₈'s and 0.7% for combined C₄'s.

### Example 2

A small-scale, glass Short-Path Distillation (SPD) apparatus is used as a first distillative unit (e.g., unit 105 in FIG. 1). The SPD provides 0.033 m² surface area for the separation. About 41 g of a mixture, prepared similar to the procedures and equipment described in Example 1, and comprising 12% acetic acid, ˜2% acetic anhydride (ACAN), 0.6% triethylamine, 1.2% butyl acetate, 33.7% n-butyl ester of 4,8-dodecanedienoic acid, 30.6% CDDT, balance non-selective products of CDDT ozonolysis, is fed to the SPD. The SPD conditions are: 110° C. evaporator, 15° C. internal condenser, 10 mmHg vacuum, 1.6 g/min averaged feed rate. The average feed residence time inside the unit is less than 3 minutes. The separation yields 25.8 g of top fraction stream and 15 g of bottom fraction stream. The bottom fraction stream is mostly the concentrated n-butyl ester of 1-oxo-4,8-dodecanedienoic acid, other non-selective products and about 1 wt % CDDT.

The top fraction stream is condensed at 15° C. (e.g., unit 111 in FIG. 1) and the condensed liquid is allowed to separate into 13.4 g of top phase and 11 g of bottom phase upon standing in a 40 cc separatory funnel (e.g., the first phase separator 131 in FIG. 1) at the room temperature of 20° C. The two phases are carefully separated from each other and GC-analyzed. The CDDT concentrates in the top phase at about 67.3% concentration. The residual CDDT in the bottom phase is about 9.2%. The bottom phase is primarily comprised of about 2:1 (wt:wt) acetic acid:ACAN. The GC indicates all triethylamine in the feed is concentrated in the bottom phase as there is no detectable amine in the top phase. However, an accurate quantification is difficult for the amine component due to poor GC peak resolution representing the acid-amine complex formed at our conditions.

### Examples 3-11

Examples 3-11 are conducted analogous to Example 2 with the operating ranges as identified in Table 2.

TABLE 2

| | | | % wt. of separated feed | | | % Recovery | |
|---|---|---|---|---|---|---|---|
| | Distillation | | Distillate | | | C₁₂ Ester | CDDT in Top |
| | Conditions | | (Top) Fraction | | | products in | Phase of |
| | Temperature | Pressure | Top | Bottom | Bottom | Bottom | distillate |
| Ex. | ° C. | mmHg | Phase | Phase | Fraction | Fraction | stream |
| 3 | 90 | 10 | 27 | 34 | 40 | 98.4 | 81.1 |
| 4 | 110 | 5 | 38 | 30 | 32 | 89.5 | 88.1 |
| 5 | 110 | 10 | 34 | 35 | 31 | 95.5 | 81.5 |
| 6 | 110 | 10 | 28 | 34 | 38 | 98.3 | 88.7 |
| 7 | 110 | 20 | 31 | 34 | 34 | 94.3 | 78.1 |
| 8 | 130 | 5 | 43 | 33 | 24 | 64.9 | 84.0 |
| 9 | 130 | 10 | 31 | 36 | 33 | 86.1 | 84.3 |
| 10 | 130 | 10 | 36 | 36 | 28 | 87.2 | 84.0 |
| 11 | 130 | 20 | 35 | 37 | 28 | 86.6 | 84.2 |

### Examples 12-17

A pilot-scale Short-Path Distillation (SPD) apparatus is used for the first separation. The SPD provides 0.06 m² surface area for the separation. The feed composition, prepared using the procedures and equipment similar to Example 1, is varied between 15-25% acetic acid, 9-37% ACAN, 2-7% triethylamine, 11-20% n-butyl ester of 4,8-dodecanedienoic acid, 22-30% CDDT, balance non-selective products of CDDT ozonolysis. Table 3 summarizes the operating ranges for Examples 12-17.

TABLE 3

| | | | | % C₁₂ Products | | wt % C₁₂ Product in . . . | |
|---|---|---|---|---|---|---|---|
| | Feed | | | split in bottom | wt % CDDT | distillate | distillate |
| | Rate | Temperature | Pressure | fraction vs. top | in Bottom | top | bottom |
| Ex. | g/min | ° C. | mmHg | fraction | Fraction | phase | phase |
| 12 | 6.5 | 110.2 | 4.9 | 93.7 | 1.7 | 2.0 | 1.5 |
| 13 | 6.6 | 110.2 | 4.9 | 94.2 | 4.0 | 2.0 | 1.5 |
| 14 | 6.7 | 120.6 | 4.9 | 85.4 | 3.9 | 4.3 | 3.2 |
| 15 | 6.4 | 120.2 | 5.0 | 93.2 | 1.1 | 4.2 | 0.0 |
| 16 | 6.3 | 121.0 | 5.3 | 82.9 | 1.3 | 5.2 | 2.6 |
| 17 | 6.0 | 120.4 | 5.0 | 80.5 | 1.2 | 2.8 | 1.8 |

### Example 18

A short-path distillation (SPD) apparatus is used for the first distillative unit (e.g., unit 105 in FIG. 1). In this example, the SPD apparatus is a sequential, two-stage arrangement with inter-stage recycle (e.g., FIG. 3); SPD First Stage and SPD Second Stage. A bottom fraction stream from SPD First Stage is fed to SPD Second Stage, while a top fraction stream from SPD Second Stage is combined with SPD First Stage feed and recycled to SPD First Stage. In this example, SPD Second Stage is used to enrich the low-boiling components of the feed entering SPD First Stage.

About 3.83 kg/hr of a process stream (e.g., stream 100 in FIG. 3), that is prepared using the procedures and equipment similar to Example 1, and comprising 19.2% acetic acid, 5.2% acetic anhydride (ACAN), 3.1% triethylamine, 1.4% butyl acetate, 23.6% n-butyl ester of 4,8-dodecanedienoic acid, 38.7% CDDT, balance non-selective products of CDDT ozonolysis, is combined with a recycled 0.96 kg/hr condensed liquid stream (e.g., stream 199 in FIG. 3) from SPD Second Stage and the combined stream is fed to SPD First Stage. SPD First Stage is operated at 5.0 mmHg and 78° C. to control n-butyl ester of 4,8-dodecanedienoic acid concentration at 0.1% in the 2.84 kg/hr top fraction stream (e.g., stream 110 in FIG. 3). SPD Second Stage is operated at 0.5 mmHg and 103° C. to control CDDT concentration at 1% in the 0.99 kg/hr bottom fraction stream (e.g., stream 150 in FIG. 3), which is the n-butyl ester of 4,8-dodecanedienoic acid product stream containing 91.2% n-butyl ester of 4,8-dodecanedienoic acid, with 2.4% 1-oxo-4,8-dodecanedienoic acid, 1.4% n-butyl ester of 1-oxo-4-octenoic acid, 1.4% dibutyl ester of 4-octenoic acid, 0.7% 4,8-dodecadienedial, 1% CDDT and the balance other C₄-C₈ impurities.

The top fraction stream from SPD First Stage is condensed at 5° C. in the condenser (e.g., unit 325 in FIG. 3). The condensed liquid stream represents the liquid stream 3 in FIG. 1. This stream is allowed to separate into two liquid phases (e.g., the phase separator 131 in FIG. 1) resulting in 1.96 kg/hr of top phase stream (e.g., stream 13 in FIG. 1) containing 71.3% CDDT and 0.88 kg/hr of bottom phase stream (e.g., stream 17 in FIG. 1) containing 66.8% acetic acid.

### Example 19

About 0.88 kg/hr of the bottom phase stream (e.g., stream 17a in FIG. 2), obtained in Example 18, is fed to a 29-stage, second distillative unit (e.g., unit 201 in FIG. 2) operating in the temperature range of 45° C. (condenser)/82° C. (head)/121° C. (base) and at reduced pressure (250 mmHg head/450 mmHg base). The feed composition includes 11.2% ACAN, 6.6% triethylamine, 66.8% acetic acid, 8.3% CDDT, 300 ppm n-butyl ester of 4,8-dodecanedienoic acid, 6.7% C₄ impurities, 0.5% C₈ impurities, 100 ppm 4,8-dodecanedial. The feed enters on the 23^{rd} stage, which provides 22 stages of rectification above the feed tray location and 7 stages of acid stripping below the feed tray location. The internal hydraulics is maintained by overhead refluxing and reboiler boil-up such as to obtain 287 kcal/hr of condenser heat duty and 315 kcal/hr of reboiler heat duty. The column overhead is obtained at 0.47 kg/hr of liquid stream (e.g., stream 43 in FIG. 2) having the composition of 96.1% acetic acid, 0.2% ACAN, 3.7% C₄ impurities (3.2% butyl acetate, 0.5% butylaldehyde). The column bottoms draw is obtained at 0.41 kg/hr rate (e.g., stream 45 in FIG. 2).

### Example 20

The bottoms draw of Example 18 (e.g., unit 226 in FIG. 2) is combined with the top phase (e.g., stream 13a in FIG. 2), and fed to the third distillative unit at a combined rate of 2.38 kg/hr. The combined feed composition includes, 8.3% ACAN, 4.9% triethylamine, 12.0% acetic acid, 62.0% CDDT, 0.1% n-butyl ester of 4,8-dodecanedienoic acid, 10.7% C₄ impurities, 1.9% C₈ impurities, 500 ppm 4,8-dodecadienedial. The third distillative unit is a 23 theoretical stage column equipped with refluxing and boil-up capabilities. The feed enters above the 11^{th} stage, which provides ten stages of rectification above the feed distributor tray and 13 stripping stages below the feed tray. The column is maintained at 45° C. (condenser)/46° C. (head)/170° C. (base) and at vacuum (25 mmHg head/30 mmHg base). The column hydraulics is maintained by overhead refluxing and reboiler boil-up such as to obtain 140 kcal/hr of condenser heat duty and 224 kcal/hr of reboiler heat duty.

The third distillative column concentrates the anhydride, and a complex of triethyamine and acid in the overhead stream (e.g., stream 49 in FIG. 2) at the rate of 0.65 kg/hr. The composition of this stream is, 30.4% ACAN, 43.7% acetic acid, 18.0% triethylamine, 7.7% C₄ impurities and 0.2% CDDT.

The column bottoms draw has the composition, of 85.3% CDDT, 11.8% C₄ impurities, 2.6% C8 impurities, 0.17% n-butyl ester of 1-oxo-4,8-dodecanedienoic acid and 600 ppm 4,8-dodecadienedial (e.g., stream 51 in FIG. 2).

### Example 21

The third distillative column bottoms stream of Example 20 is fed to a fourth distillative unit (e.g., unit 251 in FIG. 2) at the rate of 1.73 kg/hr. The fourth distillative unit is a 13 theoretical stage column, equipped with refluxing, boil-up and side draw capabilities. The liquid feed enters on the 4^{th} stage, which provides three rectification stages above the feed distribution tray and 10 stripping stages below the feed distribution tray. A liquid side draw is taken 5 stages below the feed. The column is maintained at 45° C. (condenser)/57° C. (head)/124° C. (base) and at vacuum (10 mmHg head/15 mmHg base). The column hydraulics maintains the vapor-liquid traffic according to the 102 kcal/hr of condenser heat duty and 86 kcal/hr of reboiler heat duty.

The fourth distillative column concentrates the CDDT in the side draw stream (e.g., stream 28a in FIG. 2) at the rate of 1.72 kg/hr. The composition of this stream is, 85.7% CDDT and 11.5% butyl ester of C₄ aldehyde acid, with 2.8% other C₄, C₈ and C₁₂ impurities. A small purge stream (e.g., stream 47 in FIG. 2) is taken overhead at 5.7 g/hr rate to control the low-boiling process impurities in the system. The composition of the low-boiler purge stream is, 25.8% CDDT, 71.7% C₄ dialdehyde, 1.1% butyl ester of C₄ aldehyde acid, 1% butyl acetate and 0.4% 1,8-octenedial. Another small purge stream (e.g., stream 55 in FIG. 2) is taken as the bottoms stream at 5.9 g/hr to control the mid-boiling process impurities in the system. The composition of the mid-boiler purge stream is, 25.0% CDDT, 4.1% butyl ester of C₄ aldehyde acid, 12.0% dibutyl succinate, 31.7% n-butyl ester of 1-oxo-4-octenoic acid, 0.3% 1,8-octendial, 0.8% dibutyl ester of 1,8-octenedioic acid, 7.0% 4,8-dodecadienedial, and 18.9% n-butyl ester of 1-oxo-4,8-dodecanedienoic acid.

The disclosed process, through Examples 18 to 21, obtains the contained CDDT, contained anhydride, contained amine and contained desired transformation product of the compound of formula I recovery yields of 99.0%, 99.5%, 100% and 99.7%, respectively. The contained recovery yield, for each recovered component from the feed stream 78 in FIG. 1, is defined as equal to;

$${CDDT}_{Contained Recovery Yield} = \frac{⌊ amount of CDDT in stream 28 ⌋}{[ amount of CDDT in Feed stream 78 ]} \times 100 \%$$

$${Anhydride}_{Contained Recovery Yield} = \frac{⌊ amount of Anhydride in stream 49 ⌋}{[ amount of Anhydride in Feed stream 78 ]} \times 100 \%$$

$${Amine}_{Contained Recovery Yield} = \frac{⌊ amount of Amine in stream 49 ⌋}{[ amount of Amine in Feed stream 78 ]} \times 100 \%$$

$$Desired {Product}_{Contained Recovery Yield} = \frac{⌊ amount of desired product in stream 9 ⌋}{[ amount of desired product in Feed stream 78 ]} \times 100 \%$$

In the comparative example 23, the contained CDDT recovery yield of 102% is obtained. But, the product recovery yield in example 23 is 30.4% indicating unsatisfactory separation.

### Example 22

### Mixture Separation Using Conventional Distillation and Extraction (Comparative)

A total of nine reaction batches, averaging about 126.5 g of effluent per batch, are run using the procedures and equipment described in Example 1 except the excess reagent (n-butanol) is not flashed off in all batches. About 1128.5 g of the mixture is constituted from the nine batches made. The mixture is roto-evaporated at 24 mmHg and 70° C. to remove the excess reagent and butyl acetate. About 518.8 g of the stripped material is recovered by flashing off 602.3 g of overhead distillate, which contained 294.2 g n-butanol, 122.4 g butyl acetate, 132.2 g of acetic acid, 16.4 g of CDDT and small amounts of ACAN and amine.

To the 511.1 g roto-evaporated material is added about 483 g of DI water as an extraction agent and the mixture is hand-shaken. Upon overnight standing in a separating funnel at 20-22° C., the mixture separates into 422.5 g of yellowish emulsified top layer [pH of 3.5] and 575.4 g of slightly cloudy bottom layer [pH of 4.5]. The bottom layer later is turned clear and colorless. Separation of the compound of formula III is not effective in this example.

Table 4 gives a summary of process stream compositions in terms of calculated weights for individual component using the GC analysis.

TABLE 4

| | In Feed | Roto-evaporated | Top Layer from | Bottom Layer from |
|---|---|---|---|---|
| Component | Mixture, (g) | Material, (g) | Extraction, (g) | Extraction, (g) |
| CDDT | 203.3 | 186.9 | 171.0 | 8.1 |
| n-butyl ester of 12-oxo-dodeca- | 227.6 | 227.6 | 163.0 | 7.1 |
| 4,8-dieneoic acid (the compound | | | | |
| of formula III) | | | | |
| Dodeca-4,8-diene-1,12- | 16.4 | 16.4 | 27.3 | 0.0 |
| dialdehyde | | | | |
| 12-oxo-dodeca-4,8-dieneoic acid | 2.0 | 2.0 | 1.9 | 0.0 |
| combined C₈′s | 19.3 | 19.3 | 4.1 | 0.2 |
| combined C₄′s | 7.3 | 7.3 | 0 | 7.0 |
| Acetic acid | 176.5 | 44.3 | 1.0 | 41.0 |
| ACAN | 37.7 | <15 | 0.0 | <15 |
| Triethylamine | 10.6 | <10 | 0.0 | <10 |
| n-Butanol (excess reagent) | 303.4 | <10 | <10 | 0.35 |
| Butyl acetate (from the reaction | 124.4 | <5 | <5 | 0.1 |
| between butanol and acetic acid) | | | | |
| TOTAL (grams) | 1128.5 | 518.8 | 422.5 | 575.4 |

### Example 23

### Mixture Separation Using Spinning Band Distillation (Comparative)

About 362.4 g of the water-extracted top layer, similar to Example 22 and containing 129.3 g CDDT, 180.9 g n-butyl ester of 12-oxo-dodeca-4,8-dieneoic acid (the compound of formula III), 6.5 g Dodeca-4,8-diene-1,12-dialdehyde and 3.6 g of non-selective products, is batch-fed to a lab-scale, spinning band distillation unit. About 10.5 g of liquid condensate is collected in the cold trap during the initial 3 hours of operation at approximately 300-400 mmHg column vacuum and 40-96° C. bottom temperature. The overhead temperature is 20-52° C. The cold trap material contains mostly low-boiling components, e.g., butyl acetate, butanol and residual water.

The column vacuum is further reduced to approximately 0.14 mmHg while the bottom temperature is gradually increased from about 50° C. to about 175° C. at the ramp rate of 3.3° C./hr. The overhead temperature remains below 50° C. but rapidly increases to about 100° C. near the end of the ramp. A total of seven overhead cuts [#1-7] are collected during this period and analyzed on GC. The bottom temperature is further increased from about 175° C. to about 200° C. at the ramp rate of 1.5° C./hr. The overhead temperature increases from about 100° C. to about 130° C. or so at the end of this period during which additional five overhead cuts [#8-12] are collected and analyzed on GC. Table 5 gives a summary of GC-analyzed cut compositions in weight %.

TABLE 5

| | | Cut | Cut | Cut | Cut | Cut | Cut | Cut | Cut | Cut | Cut | Cut | Cut | Bottoms |
|---|---|---|---|---|---|---|---|---|---|---|---|---|---|---|
| Component | Feed | # 1 | # 2 | # 3 | # 4 | # 5 | # 6 | # 7 | # 8 | # 9 | # 10 | # 11 | # 12 | [end-of-run] |
| CDDT | 35.4 | 86.4 | 94.7 | 80.1 | 80.3 | 75.9 | 79.0 | 6.3 | 0.5 | 0.02 | 0.0 | 0.0 | 0.0 | 0.0 |
| n-butyl ester | 50.8 | 0.0 | 0.5 | 0.0 | 0.0 | 0.14 | 1.2 | 0.0 | 8.8 | 76.5 | 84.7 | 86.0 | 85.2 | 26.2 |
| of 12-oxo- | | | | | | | | | | | | | | |
| dodeca-4,8- | | | | | | | | | | | | | | |
| dieneoic acid | | | | | | | | | | | | | | |
| Dodeca-4,8- | 1.8 | 0.3 | 0.1 | 0.0 | 0.0 | 0.0 | 0.3 | 2.5 | 18.2 | 4.6 | 0.3 | 0.4 | 0.5 | 0.0 |
| diene-1,12- | | | | | | | | | | | | | | |
| dialdehyde | | | | | | | | | | | | | | |
| Combined C8's | 0.2 | 2.3 | 0.8 | 0.7 | 0.5 | 0.5 | 0.5 | 0.6 | 0.2 | 0.0 | 0.0 | 0.0 | 0.0 | 0.0 |
| Other | 0.7 | 0.0 | 0.0 | 0.3 | 0.5 | 0.7 | 0.0 | 0.0 | 1.5 | 1.9 | 0.2 | 0.2 | 0.6 | 1.5 |
| Total (g) | 362.4 | 1.5 | 36.5 | 40.4 | 34.2 | 35.0 | 10.4 | 0.9 | 7.1 | 3.2 | 7.6 | 4.6 | 3.0 | 151.9 |

| | | | | | | | | | | | | | |
|---|---|---|---|---|---|---|---|---|---|---|---|---|---|
| Overhead | — | 0.6 | 15.1 | 8.1 | 3.7 | 3.7 | 1.0 | 1.4 | 2.3 | 4.5 | 0.8 | 1.3 | — |
| collection rate, | | | | | | | | | | | | | |
| g/hr | | | | | | | | | | | | | |

From the component balances the CDDT balance is satisfactory at 102% [g out/g in]. About 30.4% [g out/g in] is accounted for n-butyl ester of 12-oxo-dodeca-4,8-dieneoic acid out of the 184 g fed to the unit. Separation is ineffective to yield acceptable product recovery from the reaction mixture.

### Examples 24A-F

A miscible mixture comprising 10.0 g CDDT (of Table 1) and 10.0 g acetic acid is phase separated at 20-25° C. by adding a phase-separation agent to the mixture as follows:

TABLE 6

| | | Phase Separation Agent | | | | |
|---|---|---|---|---|---|---|
| | | | Wt % in | | Analysis by GC (wt %) | |
| Ex. | Mixture (wt:wt) | Agent | Mixture | # of Phases | Top Phase | Bottom Phase |
| 24A | Acetic acid:CDDT | — | — | Miscible | — | — |
| | (1.0:1.0) | | | (one phase) | | |
| 24B | Acetic acid:CDDT | D.I Water | 0.89 | Two | 12.2 g | 6.1 g |
| | (1.0:1.0) | | | | 29.6% acid | 67.7% acid |
| | | | | | 68.0% CDDT | 23.7% CDDT |
| 24C | Acetic acid:CDDT | Triethyl amine | 1.53 | Two | 15.2 g | 4.9 g |
| | (1.0:1.0) | | | | 40.3% acid | 71.2% acid |
| | | | | | 57.8% CDDT | 23.1% CDDT |
| 24D | Acetic acid:CDDT | Isopropyl amine | 1.48 | Two | 13.9 g | 6.1 g |
| | (1.0:1.0) | | | | | |
| 24E | Acetic acid:CDDT | Diisopropyl amine | 2.68 | Two | 12.6 g | 7.7 g |
| | (1.0:1.0) | | | | | |
| 24F | Acetic acid:CDDT | Pyridine | 3.61 | Two | 11.4 g | 8.5 g |
| | (1.0:1.0) | | | | | |

### Example 25

### Preparation of mixture comprising 1,5-cyclooctadiene, 8-oxo-octa-4-eneoic acid butyl ester, and acid

COD is reacted with ozone using equipment and procedure described in Example 1. The reactor is charged with 35.0 g of COD (0.324 mole) and 65.0 g (0.878 mole) of 1-butanol. A flow of 21% O₂ in Argon is fed to the ozone generator followed by flowing to an ozone monitor. A flow of 21 pm is set on the ozone generator that flows to the ozone monitor. A steady state (20-30 min) concentration of 33.0 g ozone/m³ in Argon is measured continuously on the monitor. After ˜15 min at steady state, the feed ozone in Argon is diverted to the reactor. The jacketed reactor containing, a mechanical stirrer, a tube for the ozone addition, an exit gas fitting and a fourth port for addition of reagents and sampling with a thermocouple is maintained at minus 5° C. The gas is flowed through the reactor followed by passing through a Dry Ice cold trap followed by a scrubber containing 66.0 g tetradecane. The run time is 141 min. The ozone generator is then turned off and nitrogen is then passed into the reactor for 5 min to remove any residual ozone.

When the reaction is complete, un-reacted 1-butanol is removed under high vacuum at <50° C. (max) and ˜462-472 mtorr. The reactor is warmed to 25° C. followed by the addition of 32.0 g acetic anhydride (0.313 mole) and run for 15 min. Triethylamine (12.0 g, 0.118 mole) is next added while keeping the temperature below 25° C. After the complete addition the reaction is run for 120 min. The conversion of 1,5-cyclooctadiene is 81% (92% accounted for with the remaining lost in the off gas). Selectivity to 8-oxo-octa-4-eneoic acid butyl ester is 84.1% along with selectivities of 7.5%, 6.1% and 3.1% to 4-oxo-succinic acid butyl ester, dibutyl succinate and 1,8-octadial, respectively.

### Example 26

### Separation of mixture comprising COD, 8-oxo-octa-4-eneoic acid butyl ester, acid

About 109.4 g of reaction effluent, prepared similar to the Example 25 procedure, is fed to the SPD apparatus as described in Example 2. The feed comprises of 20.3 g of COD, 18.0 g of 8-oxo-octa-4-eneoic acid butyl ester, 1.2 g of dibutyl succinate, 0.6 g of 4-oxo-succinic acid butyl ester, 0.4 g of 1,8-octadial, and other organic impurities. The SPD conditions are: 60-72° C. evaporator, 20° C. internal condenser, 20 mmHg vacuum. The separation yields 47.1 g of top fraction stream and 57.6 g of bottom fraction stream. The bottom fraction stream is mostly 8-oxo-octa-4-eneoic acid butyl ester with other non-selective products and about <5.0 wt % COD. The top fraction stream comprises of mostly COD, acid, ACAN and triethylamine.

### Examples 27A-D

A miscible mixture comprising 10.0 g COD and 10.0 g acetic acid is phase separated at 20-25° C. by adding a phase-separation agent to the mixture, as shown in Table 7.

TABLE 7

| | | Phase Separation Agent | | | | |
|---|---|---|---|---|---|---|
| | | | Wt % in | | Analysis by GC (wt %) | |
| Ex. | Mixture (wt:wt) | Agent | Mixture | # of Phases | Top Phase | Bottom Phase |
| 27A | Acetic acid:COD | — | — | Miscible | — | — |
| | (1.0:1.0) | | | (one phase) | | |
| 27B | Acetic acid:COD | D.I Water | 2.1 | Two | 11.9 g | 6.4 g |
| | (1.0:1.0) | | | | 33.6% acid | 64.7% acid |
| | | | | | 65% COD | 34% COD |
| 27C | Acetic acid:COD | Triethyl amine | 5.7 | Two | 9.7 g | 11.3 g |
| | (1.0:1.0) | | | | 25% acid | 56.9% acid |
| | | | | | 72% COD | 32% COD |
| 27D | Acetic acid:COD | Triethyl amine | 19.5 | Two | 6.0 g | 17.9 g |
| | (1.0:1.0) | | | | 1.7% acid | 44.5% acid |
| | | | | | 96% COD | 26% COD |

### Example 28

### Mixture Separation at Large Scale

A large-scale SPD unit, such as a 12-inch diameter single-stage molecular still of Pope Scientific Inc., is used for the separation. The heated chamber in the SPD unit is about 1-ft diameter [D] and about 4-ft long [L], i.e., of the aspect ratio [L/D] of about four. The heated evaporative surface area of this unit is about 1.0 m². The SPD scale-up is about 30× at the feed flux of about 1500 g/min/m² as compared against the flux of 48.5 g/min/m² in Example 2.

A homogeneous mixture comprising CDDT, n-butyl ester of 12-oxo-dodeca-4,8-dieneoic acid, acid, ACAN, amine and other organic species is fed to the unit at 90-100 kg/hr rate. The feed mixture is prepared using the equipment and procedures described in Example 1. The SPD is operated at conditions similar to those given in Examples 2-17. The residence time of the material through the unit is 5 minutes or less. The feed is separated into a top fraction stream and bottom fraction stream.

The bottom fraction stream is concentrated in the desired butyl ester product. The top fraction stream separates upon cooling to 15° C. into a CDDT-rich light phase and an acid-rich heavy phase. The light:heavy phase flow split fraction of the top fraction stream is about 40:60 (wt/wt). The separated individual phases are distillatively processed to obtain recyclable components.

### Example 29

### Mixture Separation at Industrial Scale

An industrial-scale SPD unit, having the 10 m² heated evaporative surface area, such as an Incon Process Systems short-path evaporator unit, is used for the separation. The vertical heated chamber in the SPD unit is about 3.5 ft diameter [D] and about 12.5 ft long [L] with an aspect ratio [L/D] of about 3.6. The vapor chamber is equipped with an internal surface condenser for condensing low-boiling vapor. The SPD scale-up is about 10× at the feed flux of about 14 kg/min/m² as compared against the flux in Example 28.

The liquid feed, similar to that of Example 28 and comprising CDDT, n-butyl ester of 12-oxo-dodeca-4,8-dieneoic acid, acid, ACAN, amine and other organic species, is fed to the unit in the 6-10 tons/hr range. The feed mixture is prepared using the equipment and procedures described in Example 1. The SPD is operated at conditions similar to those referenced in Example 28. The residence time of the material through the unit is less than 10 minutes. The SPD separates the feed into a top fraction stream and bottom fraction stream.

The bottom fraction stream is concentrated in the desired butyl ester product. Upon cooling to 10° C. the top fraction stream quickly separates into a CDDT-rich light phase and an acid-rich heavy phase. The light:heavy phase flow split fraction of the top fraction stream is about 30:70 (wt/wt).

### Example 30

In an embodiment of the present disclosure, a pilot-scale Short-Path Distillation (SPD) apparatus having 0.03 m² surface area and similar to the one described in Examples 12-17, is used for the separation. The hydrocarbon feed, prepared according to the Example 1 method, is subjected to two sequential stages (or passes) through this SPD unit. The per-pass separation conditions are adjusted to obtain a desired level of separation. Alternatively, the same operation can be performed in multiple SPD units connected in a series combination.

Cooling in the SPD internal condenser is done with normal city-supplied cooling water in the 20-28° C. range. Heating is supplied by an electric blanket wrapped around the glass. Two thermocouples are used in the measurement of process temperature; an upper thermocouple as a sensor for the temperature controller and a lower temperature close to the evaporation section. The lower temperature reading is generally 4-7° C. below the upper control point. Pressure measurement is made by calibrated pressure transducers in the vacuum system. Most flow rates are determined manually, i.e., at the end of the run, the flow rate recorded is determined by the total time required and the total feed consumed, expressed in g/min. Distillation times for the first stage process are normally in a range up to 12-17 hrs. Distillation times for the second stage process are normally in the range up to 12-19 hrs.

Table 8 below represents the first stage or first pass (SPD-1) and second stage or second pass (SPD-2) performance data according an embodiment of the present disclosure. In the table, “Bx Number” means individual runs 13 through 27 and “C12 OBE” means the desired C₁₂ butyl ester product (n-butyl ester of 4,8-dodecanedienoic acid). The temperature represents the upper thermocouple reading used as a control temperature.

TABLE 8

| | | | | | | | | | | | | | Resultant |
|---|---|---|---|---|---|---|---|---|---|---|---|---|---|
| | | | | | | | | | | | | Analysis | Product |
| | | | | | Distil- | | | | | Distil- | Product | of C12 | Mass C12 |
| | temper- | pres- | | Mass | lation | temper- | pres- | | Mass | lation | Yield | OBE Purity | OBE in feed |
| Bx | ature | sure | Flow | processed | time | ature | sure | Flow | processed | time | (grams) | (wt. %) | (grams) |
| Num- | C. | mm Hg | g/hour | grams | (hours) | C. | mm Hg | g/hour | grams | (hours) | Product Feedstock | | |
| ber | SPD-1 | | | | | SPD-2 | | | | | to SPD-3 Process | | |
| 13 | 72-78 | 4.5 | 122 | 1827.6 | 15.0 | 100-109 | 0.45 | 78 | 1111 | 14.2 | 613 | 69.6% | 427 |
| 14 | 71-76 | 4.5 | 117 | 1836.7 | 15.7 | 101-108 | 0.45 | 77 | 1158 | 15.0 | 630 | 72.1% | 454 |
| 15 | 71-78 | 4.3-4.8 | 140 | 1839.1 | 13.1 | 92-99 | 0.25 | 87 | 1140 | 13.1 | 645 | 71.3% | 460 |
| 16 | 72-80 | 4.3-4.5 | 138 | 1831.5 | 13.3 | 102-106 | 0.5 | 86 | 1153 | 13.4 | 613 | 67.3% | 413 |
| 17 | 72-80 | 4.5 | 126 | 1843.9 | 14.6 | 100-110 | 0.5 | 88 | 1195 | 13.6 | 624 | 71.5% | 446 |
| 18 | 72-77 | 4.5 | 127 | 1843.0 | 14.5 | 101-110 | 0.5 | 94 | 1130 | 12.0 | 629 | 68.0% | 428 |
| 19 | 71-78 | 4.5 | 138 | 1840.0 | 13.3 | 103-110 | 0.45 | 86 | 1113 | 12.9 | 625 | 71.4% | 446 |
| 20 | 71-78 | 4.7 | 136 | 1838.0 | 13.5 | 103-110 | 0.44 | 86 | 1177 | 13.7 | 629 | 72.7% | 457 |
| 21 | 72-76 | 4.5 | 138 | 1836.7 | 13.3 | 106-113 | 0.46 | 83 | 1172 | 14.1 | 615 | 66.8% | 411 |
| 22 | 70-78 | 4.5 | 130 | 1850.3 | 14.2 | 104-108 | 0.45 | 89 | 1114 | 12.5 | 664 | 71.4% | 474 |
| 23 | 68-76 | 4.5 | 147 | 1849.0 | 12.6 | 101-110 | 0.45 | 43 | 1229 | 28.6 | 852 | 61.3% | 522 |
| 24 | 72-80 | 4.3 | 106 | 1834.0 | 17.3 | 103-110 | 0.43 | 65 | 1108 | 17.0 | 576 | 68.0% | 392 |
| 25 | 70-78 | 4.5-4.7 | 117 | 1843.0 | 15.8 | 105-111 | 0.46 | 66 | 1110 | 16.8 | 664 | 71.6% | 475 |
| 26 | 72-77 | 4.5 | 106 | 1860.0 | 17.5 | 102-110 | 0.43 | 61 | 1150 | 18.9 | 649 | 71.9% | 467 |
| 27 | 73-78 | 4.5-4.8 | 113 | 1344.0 | 16.3 | 100-109 | 0.47 | 68 | 1150 | 16.9 | 630 | 71.9% | 453 |

The first-stage SPD-1 separation yields a first-stage top fraction stream rich in a mixture of acetic acid, ACAN and trimethylamine. The first-stage bottom stream contains CDDT, the desired C₁₂ butyl ester (n-butyl ester of 4,8-dodecanedienoic acid) product, and non-selective C₄, C₈, C₁₂ components, which is fed to the second SPD-2 stage. The second-stage separation yields a second-stage top fraction stream rich in CDDT and may contain less than 10% C₁₂ butyl ester product. This CDDT rich stream is taken to another separation column and the CDDT is recovered for recycle. The second-stage bottom stream is concentrated in the C₁₂ ester products, which may be optionally fed to the third SPD-3 stage for further refinement. Alternatively, this concentrated ester stream may be used in downstream processing.

### Example 31

A pilot-scale Short-Path Distillation (SPD) apparatus, similar to the one described in Examples 12-17, is used for the third-stage or third pass (SPD-3) separation. The SPD provides 0.06 m² surface area for each stage of the separation. Using several batches of the ester product stream generated according to the Example 30 [Table 8 Bx Nos. 13-27], a composite feed is prepared and GC-measured to contain (by weight relative to the total); <0.5 TEA, <0.7 HOAc, 0.4 ACAN, 0.1 BuOAc, 0.1-2 CDDT, 70-72 n-butyl ester of 4,8-dodecanedienoic acid, 10-12 combined butyl ester of C₅ aldehyde, C₈ dibutylester, Dodeca-4,8-diene-1,12-dialdehyde, and about 12-18 high boiling impurities that are not identified in the GC analysis.

The third stage (SPD-3) conditions are adjusted such that the ester product may be concentrated in the overhead by rejecting the high-boiling impurities in the bottom. Table 9 represents various conditions tested in the third stage for the ester product recovery in the overhead.

TABLE 9

| | Thrid-pass SPD Conditions | | | Third-pass SPD Overhead Distillate Recovery | | | |
|---|---|---|---|---|---|---|---|
| | Feed Rate | Evap. Temp | Vacuum | % Recovery | % purity | % recovery | % Recovery |
| No. | g/hr | Deg. C. | mmHg | Low Boiler | Ester product | High Boiler | Total |
| 30a | 153 | 122 | 0.047 | 87.1 | 81.9 | 2.9 | 90.0 |
| 30b | 155 | 123 | 0.057 | 101.1 | 82.5 | 3.4 | 104.5 |
| 30c | 137 | 102 | 0.034 | 77.6 | 84.7 | 9.4 | 87.0 |
| 30d | 144 | 117 | 0.036 | 89.4 | 84.3 | 3.8 | 93.2 |
| 30e | 158 | 132 | 0.054 | 91.8 | 81.6 | 1.6 | 93.4 |
| 30f | 248 | 119 | 0.051 | 83.7 | 87.2 | 8.9 | 92.6 |
| 30g | 83 | 87 | 0.039 | 49.7 | 89.2 | 45.6 | 95.3 |
| 30h | 91 | 133 | 0.040 | 92.1 | 84.0 | 1.8 | 93.9 |
| 30i | 148 | 118 | 0.044 | 94.9 | 86.4 | 4.6 | 99.5 |
| 30j | 91 | 117 | 0.046 | 92.8 | 83.5 | 3.7 | 96.5 |
| 30k | 331 | 143 | 0.15 | 88.6 | 81.95 | 5.7 | 94.3 |
| 30l | 284 | 145 | 0.13 | 95.9 | 85.12 | 4.3 | 100.2 |
| 30m | 290 | 145 | 0.13 | 93.9 | 84.34 | 5.4 | 99.3 |
| 30n | 240 | 147 | 0.12 | 97.4 | 84.31 | 2.9 | 100.3 |
| 30o | 303 | 148 | 0.11 | 94.9 | 85.63 | 6.6 | 101.5 |
| 30p | 277 | 143 | 0.12 | 93.9 | 85.30 | 5.4 | 99.3 |
| 30q | 242 | 143 | 0.11 | 95.3 | 85.74 | 3.8 | 99.1 |

The ester product composition in the third-stage (SPD-3) distillate is represented in Table 10 below.

TABLE 10

| | | Average |
|---|---|---|
| Component | Range (% by wt.) | (% by wt.) |
| n-butyl ester of 4,8-dodecanedienoic acid | 82.0-85.7 | 84.6 |
| Dodeca-4,8-diene-1,12-dialdehyde | 4.2-4.9 | 4.7 |
| C₈ dibutyl ester | 4.0-4.4 | 4.2 |
| Butyl ester of C₈ aldehyde | 2.3-3.2 | 2.8 |
| C₈ dialdehyde | 0-0.1 | 0.06 |
| CDDT | 1.6-2.3 | 1.9 |
| C₄ diester/butyl ester of C₄ aldehyde | 0.2-0.9 | 0.5 |
| ACAN | 0.0-0.2 | 0.1 |
| Total % Accounting | 95.3-100.2 | 98.9 |

### Example 32

A 10-liter batch of liquid feed is prepared using the pure components. The liquid mixture batch is gently stirred and has the following composition upon completion.

TABLE 11
Acetic acid purge column feed composition

| Component | Quantity (g) | wt % |
|---|---|---|
| HOAc | 6729.0 | 67.9 |
| Triethyl Amine | 803.0 | 8.1 |
| ACAN | 890.7 | 9.0 |
| BuOAc | 296.9 | 3.0 |
| CDDT | 1189.4 | 12.0 |

The above liquid mixture is gently agitated for one hour and allowed to settle for about 30 minutes. Two distinct liquid phases are formed upon standing. The bottom phase (˜85.8 wt % of total mixture) is carefully decanted. The remaining top phase is added to a separatory funnel for further decanting of any remaining lower phase. After several careful additions of the top phase, a small quantity of a third lighter liquid phase is observed. The larger middle phase is carefully decanted and recovered followed by collection of the top-most liquid layer. The middle liquid layer accounts for approximately 13.7 wt % while the top layer is approximately 0.5 wt %, both relative to the initial liquid feed. Each monophasic layer sample is GC analyzed. Table 12 represents the GC analyses of the three liquid phases as recovered above.

TABLE 12
Normalized compositions of distillation feed liquid layers

| | Concentration (Normalized wt %) | | | | |
|---|---|---|---|---|---|
| Layer | Triethyl Amine | HOAc | ACAN | BuOAc | CDDT |
| Top | 0.68 | 16.42 | 1.59 | 3.39 | 77.93 |
| Middle | 8.37 | 66.39 | 6.79 | 3.09 | 15.36 |
| Bottom | 8.12 | 70.48 | 7.03 | 3.06 | 11.31 |

The small top layer is enriched in CDDT while the middle and bottom layers are similar. In all three layers, the concentration of acetic acid is in large excess relative to triethylamine. The feed layer concentrations are re-calculated in Table 13 to determine the amounts of 3:1 (molar) acetic acid:triethylamine complex and un-complexed or “free” acetic acid.

TABLE 13
Corrected compositions of distillation feed liquid layers

| | Concentration (Normalized wt %) | | | | |
|---|---|---|---|---|---|
| | 3:1 Complex of | Free | | | |
| Layer | HOAc:TEA | HOAc | ACAN | BuOAc | CDDT |
| Top | 1.89 | 15.21 | 1.59 | 3.39 | 77.93 |
| Middle | 23.28 | 51.48 | 6.79 | 3.09 | 15.36 |
| Bottom | 22.59 | 56.01 | 7.03 | 3.06 | 11.31 |

### Example 33

A pilot-scale column is arranged for batch rectification from a 12-liter round bottom glass pot fitted with a 2.2 kW heating mantle. The column consists of two five foot vacuum-insulated lengths of mirrored 2-inch inside diameter (I.D) columns containing high efficiency Koch-Glitsch structured packing yielding 41 total theoretical stages (N) including pot/reboiler. Vapor leaving the column is condensed in a thermostatically-jacketed glass condenser cooled by a recirculating water/glycol mixture from a high capacity chiller. Reflux ratio is controlled by an electromagnet-driven cup-sealed splitter and distillate is condensed and collected in a secondary condenser-receiver. The system can be operated from high vacuum (˜25 mmHg absolute) to slightly above ambient pressure (<3 psig) using a regulated vacuum/N₂ backfill pressure control system. The temperatures of the pot (N≈41), column mid-point (N≈22), column top (N≈2) and condenser are DCS-monitored and recorded using centrally-placed thermocouples.

The bottom layer (˜8.5 kg), recovered according to the Example 32 method, is charged to the pot. Subsequently, the column head pressure is reduced to 250 mmHg vacuum, the condenser temperature is set to 25° C. and boil-up is established under total reflux. Under these conditions, the pot temperature is 93.5° C. and the column mid- and top-point temperatures are 82.8° C. and 82.5° C., respectively. At this point, the reflux splitter is started at a reflux ratio of 3.0 and distillate collection is initiated. Distillate fractions of approximately 100 or 200 mL are collected and weighed over the course of the campaign. Small (1-2 cc) samples are collected from each fraction and analyzed by GC.

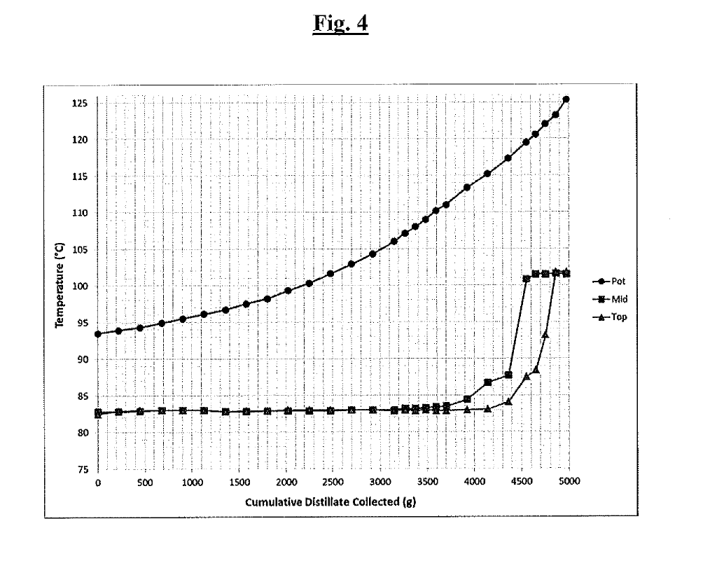

FIG. 4 represents the measured column temperature profile at 250 mmHg head pressure and reflux ratio of 3.0.

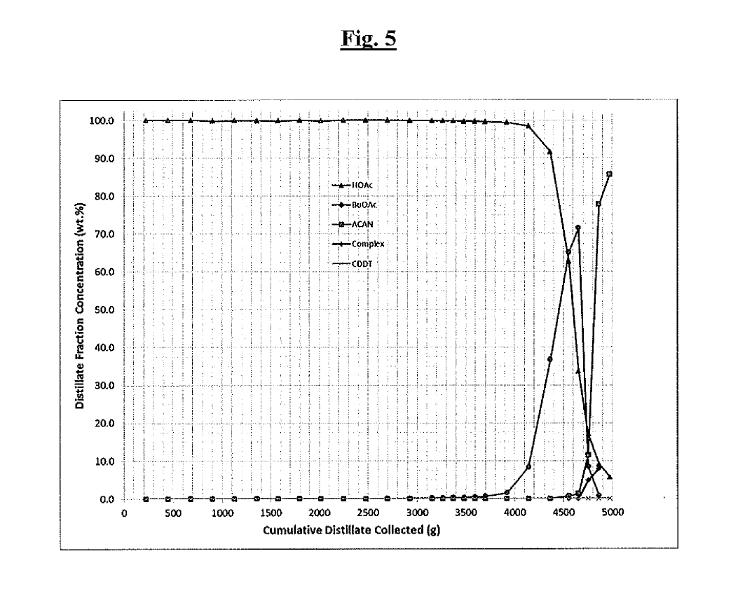

FIG. 5 represents the measured column distillate fraction compositional profiles at 250 mmHg head pressure and reflux ratio of 3.0.

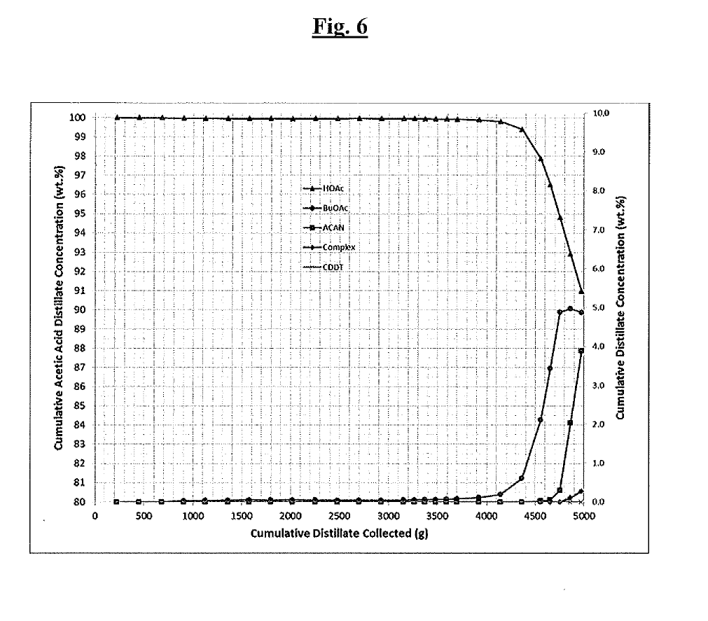

FIG. 6 represents the cumulative column distillate fraction compositional profiles at 250 mmHg head pressure and reflux ratio of 3.0.

Analyses of liquid samples are carried out by gas chromatography using an Agilent 6890A GC with a 30 m×0.25 mm×1.0 μm DB-1701 capillary column and flame ionization detector. All samples are diluted 100:1 by mass prior to analysis with an internal standard solution containing 5 wt % N-methylpyrrolidone in m-xylene solvent. Peak areas are calibrated in separate experiments with standards of known concentration and referenced to the internal standard response. The chromatograms show excellent baseline resolution and quantitation of butyl acetate, ACAN and CDDT.

### Example 34

In order to investigate the recovery of free un-complexed triethylamine and acetic acid, standard samples are prepared with molar ratios of acid:amine of 5:1 and 3:1 at 100:1 dilution. Table 14 compares the expected concentrations and the analyzed concentrations of TEA and HOAc. In the table, “Xᵢ” represents the concentration by weight of the component “i”.

TABLE 14
Comparison of acid and amine accounting in standard samples

| | | |
|---|---|---|
| | Expected | Measured |
| acid:amine | (wt %) | (Normalized wt %) |

| ratio (molar) | X_{HOAc} | X_{TEA} | X_{HOAc} | X_{TEA} |
|---|---|---|---|---|
| 3:1 | 64.03 | 35.97 | 63.01 | 36.99 |
| 5:1 | 74.79 | 25.21 | 73.83 | 26.17 |

### Example 35

A total of approximately 5 kg of the initial 8.5 kg charge according to the Example 33 feed is distilled over a total run time of nearly 7.5 hrs. At this point, the column is returned to total reflux and allowed to cool to ambient temperature. Once all liquid hold-up in the column had drained to the pot, the reboiler contents are collected, revealing a distinct two layer liquid mixture. The top layer is approximately 20 vol % of the remaining pot inventory with the larger lower layer making up the volume balance. A small sample from each separate phase is GC-analyzed and is represented in Table 15 below.

TABLE 15
Distillation Heel Liquid Layers

| | Concentration (Normalized wt %) | | | | |
|---|---|---|---|---|---|
| Layer | HOAc:TEA Complex | HOAc | ACAN | BuOAc | CDDT |
| heel top | 1.64 | 1.15 | 0.53 | 0.00 | 96.69 |
| heel | 70.08 | 4.15 | 15.25 | 0.00 | 10.52 |
| bottom | | | | | |

The remaining pot inventory is highly enriched in the 3:1 acid:amine complex, ACAN and CDDT. The top heel layer is highly concentrated in CDDT, with the balance comprising small amounts of 3:1 acid:amine complex, free acid and ACAN. The larger lower layer contains the bulk of the 3:1 acid:amine complex with the balance having a small amount of free acid and the remaining unrecovered ACAN and CDDT. Neither layer contains butyl acetate as verified by the GC analysis.

All patents, patent applications, test procedures, priority documents, articles, publications, manuals, and other documents cited herein are fully incorporated by reference to eh extent such disclosure is not inconsistent with this invention and for all jurisdictions in which such incorporation is permitted.

When numerical lower limits and numerical upper limits are listed herein, ranges from any lower limit to any upper limit are contemplated.

While the illustrative embodiments of the invention have been described with particularity, it will be understood that various other modifications will be apparent to and may be readily made by those skilled in the art without departing from the spirit and scope of the invention. Accordingly, it is not intended that the scope of the claims hereof be limited to the examples and descriptions set forth herein but rather that the claims be construed as encompassing all the features of patentable novelty which reside in the present invention, including all features which would be treated as equivalent thereof by those skilled in the art to which the invention pertains.

We claim:

1. A method for separating a mixture comprising a compound of formula I: 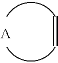
and a compound of formula III: 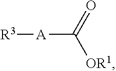
comprising distilling the mixture, wherein A is a C₆-C₁₀ alkene chain with at least one double bond, R¹ is a C₁-C₁₀ alkyl, and R³ is an oxygen-containing functional group.

2. The method of claim 1, wherein the mixture is a crude mixture from ozonolysis of the compound of formula I.

3. The method of claim 1, wherein the mixture further comprises an acid.

4. The method of claim 3, wherein the acid is an acetic acid.

5. The method of claim 3, wherein the mixture is distilled through at least one flash distillation.

6. The method of claim 5, wherein the flash distillation is a short path distillation.

7. The method of claim 5 wherein at least one flash distillation is a single-stage flash distillation.

8. The method of claim 5, wherein at least one flash distillation is a two-stage flash distillation.

9. The method of claim 5, wherein at least one flash distillation is a three-stage flash distillation.

10. The method of claim 8, wherein the first flash distillation is to remove an overhead comprising the acid and the compound of formula I from the mixture.

11. The method of claim 10, wherein the second flash distillation is to further remove the compound of formula I from the mixture.

12. The method of claim 11, wherein the bottom stream after the second flash distillation contains more than 70% the compound of formula III.

13. The method of claim 9, wherein the overhead stream after the third flash distillation contains more than 70% the compound of formula III.

14. The method of claim 10, wherein the acid and the compound of formula I in the overhead is separable by liquid-liquid phase separation.

15. The method of claim 3, wherein the mixture further comprises an acid anhydride and an amine.

16. A method of separating a miscible mixture comprising a compound of formula I: 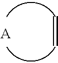
and an acid into two phases, comprising adding a phase-separation agent to the mixture, wherein A is a C₆-C₁₀ alkene chain with at least one double bond.

17. The method of claim 16, wherein the acid is acetic acid.

18. The method of claim 16, wherein the phase-separation agent is selected from the group consisting of an amine, water, a nitrile, a hydrocarbon, and mixtures thereof.

19. The method of claim 18, wherein the phase-separation agent is an amine or water.

20. The method of claim 19, wherein the amine can be in a free base, a salt or a complex form.
